(19) United States
(12) Patent Application Publication
(10) Pub. No.: US 2023/0130734 A1
(43) Pub. Date: Apr. 27, 2023

(54) CELL CULTURE FEEDING DEVICE

(71) Applicant: Regenerative Research Foundation, Rensselaer, NY (US)

(72) Inventors: Taylor Bertucci, Castleton, NY (US); Steven Lotz, Colonie, NY (US); Sally Temple, Rensselaer, NY (US); Jeffrey Stern, Slingerlands, NY (US)

(21) Appl. No.: 17/973,358

(22) Filed: Oct. 25, 2022

Related U.S. Application Data
(60) Provisional application No. 63/272,461, filed on Oct. 27, 2021.

(51) Int. Cl. C12N 5/071; C12N 5/074

(52) U.S. Cl. CPC C12N 5/0602; C12N 5/0696; C12N 2533/40; C12N 2501/11; C12N 2501/115; C12N 2539/00

(57) ABSTRACT

A non-degradable device for use in controlled feeding of mammalian cell cultures including by way of example cultures of stem cells such as induced pluripotent stem cells (iPSCs). Methods of making and using the device are also disclosed.

### RELATED APPLICATIONS

This application claims priority to U.S. Provisional Application No. 63/272,461, filed on Oct. 27, 2021, the entire contents of which is hereby referenced in its entirety.

### FIELD OF INVENTION

This disclosure pertains to devices for feeding and culturing mammalian cells. Disclosed herein is a non-degradable device for use in controlled feeding mammalian cell cultures including by way of example cultures of stem cells such as induced pluripotent stem cells (iPSCs).

### BACKGROUND

Growth factors (GFs) such as for example FGF2, FGF4, FGF8, FGF18, WNT1, WNT3A, EGF, BDNF, GDNF, NT3, TGFb1, TGFb3, BMP2, BMP4, PDGFBB, PDGFAA, IGF1, VEGFA, VEGFC, SHH, cAMP, LIF, NRG1, SCF, ACTIVIN A, IL1b, IL2, IL6, IL7, IL12, IL15, IL21, IL34, IFNα, IFNγ, TAU, ABETA, A-SYNUCLEIN or modified versions and other cell culture media additives such as fetal bovine serum (FBS) are needed to maintain cells in cell cultures, to promote cell proliferation to expand cultures, or to guide the development or differentiation of cells into desired cell or tissue products.

The term “growth factor” refers to a naturally occurring, endogenous or exogenous protein, or recombinant protein, capable of stimulating cell growth, survival and inhibiting and/or stimulating differentiation of cells, such as e.g., stem or progenitor cells. The term “growth factor” also can encompass lipid, chemical, and other non-protein agents, e.g., small molecules that are capable of stimulating cell growth, survival and inhibiting and/or stimulating cell differentiation or mixtures of these as found in FBS. In certain embodiments, the term “growth factor” refers to any polypeptide or other agent that is capable of stimulating cell growth, survival and inhibiting or stimulating cell differentiation, e.g., when present in effective amounts in a stem or progenitor cell culture. Growth factor polypeptides referred to herein include both naturally occurring and recombinant proteins, which may be either endogenous or exogenous to the cells being cultured. In addition, a growth factor may be a synthetic protein, such as a fusion or other protein construct or a chemical modification of the amino acid sequences derived from a naturally occur-ring growth factor or other protein. Such growth factors may be used in combination, to produce, e.g., an additive or synergistic effect, according to the present methods.

It is well known in the art that GFs generally have short half-lives. The labile nature of GFs means that the cells in culture require frequent, often daily, addition of GFs to the culture media to sustain the level of GFs needed to successfully maintain cells or to sustain cell growth and development or cell differentiation over time. Frequent feeding schedules subject cells to fluctuating levels of GF signaling due to GF half-lives on the order of hours to minutes. Because different growth factors have different rates of decay, the ratio of different GFs in the culture medium varies. The resulting fluctuations in GF levels and GF ratios impede effective cell culture while frequent manual replenishment of GFs results in high medium usage and increased labor. It is desirable that cell culture research or clinical use occur under controlled GF conditions, and this is not achieved with labile GFs. These practical challenges to creating quality cell cultures can be overcome by a device that provides steady, controlled GF levels.

Systems that degrade in an aqueous environment (“degradable systems”) have been employed to provide controlled-release GF to overcome many of the limitations that soluble GFs pose to effective cell culture. One example is PLGA encapsulated fibroblast growth factor-2 (FGF2) microbeads. PLGA, PLG, or poly(lactic-co-glycolic acid) is a copolymer which is used in a host of Food and Drug Administration (FDA) approved therapeutic devices, owing to its biodegradability and biocompatibility. Biodegradable “microspheres” and “millicylinders” prepared from biocompatible polyesters of glycolic and lactic acids (“PLGA”). are known for delivering protein drugs to patients, and PLGA millicylinders encapsulated with recombinant human FGF2 (also known as “basic fibroblast growth factor or “bfgf” have been described by Zhu et al. (Nature Biotechnology (2000) 18:52-57) for such applications. Olaye et al. (European Cells and Materials (2008) 16 (Suppl. 3):86) disclose that “PLGA microspheres have been extensively used for the sustained delivery of growth factors for embryonic stem cell differentiation,” The value of such degradable controlled release GF formulations, however, is limited by inability to readily remove these formulations (microbeads) from the cell cultures. For example, degradable beads stick to cells in the culture vessel and are difficult to fully wash away. Other degradable feeding formats such as films become friable as they resorb over time making clean removal from the culture difficult, leaving breakdown products. Residual degradable GF formulations are problematic because they impair the ability to control the amount of GF in the medium. Furthermore, residual degradable GF formulations impede the desired differentiation of cells that require a clean exchange of one GF environment to another. The ability to completely remove one or more GFs from the medium to leave only a negligible (not enough to provide detectable bioactivity) trace of the GF can be of great importance in some instances.

Another disadvantage of current biodegradable cell culture additives such as beads or films is their interference with imaging of the cells.

Another aspect to maintaining quality cell cultures is removing unwanted factors from the cell culture medium. Currently, this is achieved by frequent medium exchanges, once again resulting in a high and expensive level of medium usage as well as increased labor. Using the devices disclosed herein, unwanted growth factors that are desirably removed from the cell culture medium can be sequestered and removed from culture medium, obviating frequent medium changes and costly feeding.

### SUMMARY

The present disclosure describes a new platform technology that addresses the above limitations by providing a cell culture feeding device that is not degradable in aqueous environments and which provides a controlled level of GF release.

The feeding devices disclosed herein can be readily removed from, and installed in, cell culture media without requiring the medium to be exchanged or refreshed.

In some embodiments the cell culture feeding device comprises a hydrogel polymer support, a plurality of microbeads within the support, the microbeads carrying cellular growth factors (GFs).

In some embodiments the hydrogel polymer support is non-degradable.

In some embodiments the support is biologically acceptable material.

In some embodiments wherein the microbeads carry at least one GF member selected from the group consisting of FGF2, FGF4, FGF8, FGF18, WNT1, WNT3A, EGF, BDNF, GDNF, NT3, TGFb1, TGFb3, BMP2, BMP4, PDGFBB, PDGFAA, IGF1, VEGFA, VEGFC, SHH, cAMP, LIF, NRG1, SCF, ACTIVIN A, IL1b, IL2, IL6, IL7, IL12, IL15, IL21, IFNα, IFNγ, TAU, ABETA, A-SYNUCLEIN or modified versions.

In some embodiments the support includes particulate elements carrying GFs.

In some embodiments the support is transparent.

In some embodiments the microbeads are degradable.

In some embodiments the microbeads carry small molecule compounds.

In some embodiments the microbeads comprise living cells.

In some embodiments a tether is attached to the support.

In some embodiments the support comprises magnetic particles.

In some embodiments the support includes a color.

In some embodiments the support contains gas bubbles to enable the support to float at or near the surface of cell culture media.

In some embodiments the support comprises a color, magnetic particles and one or more GFs.

Some embodiments comprise a biologic cell culture medium containing the support and microbeads carrying a GF.

Some embodiments provide a method of feeding a cell culture which comprises depositing an inert hydrogel polymer support into a cell culture media, the support carrying microbeads bearing one or more cellular GFs, the GFs being continuously released into the cell culture medium at a controlled rate over a period of time.

In some embodiments the GFs used in the method of feeding are selected from the group consisting of FGF2, FGF4, FGF8, FGF18, WNT1, WNT3A, EGF, BDNF, GDNF, NT3, TGFb1, TGFb3, BMP2, BMP4, PDGFBB, PDGFAA, IGF1, VEGFA, VEGFC, SHH, cAMP, LIF, NRG1, SCF, ACTIVIN A, IL1b, IL2, IL6, IL7, IL12, IL15, IL21, IFNα, IFNγ, TAU, ABETA, and A-SYNUCLEIN or modified versions thereof.

In some embodiments the support is removed from the cell culture with a tether attached to the support.

In some embodiments the culture media contains a plurality of supports, each support is colored and bears a different GF and all of the supports have a different color.

In some embodiments the method of feeding includes depositing a plurality of colored supports into the cell culture medium.

In some embodiments the support comprises an open lattice structure.

In some embodiments the lattice structure comprises open pores.

Some embodiments include a method of making a feeding device for cell cultures by preparing a solution containing a biologically acceptable polymer and a quantity of microspheres bearing at least one GF, dispensing droplets of the solution onto a surface, and exposing the droplets to actinic radiation to form a hydrogel support.

In some embodiments, the GFs are released in a cell culture over a period of time.

In one implementation the support is preferably in the shape of a disc, square, triangle or rectangle or comprises a free form arrangement.

In one embodiment the support is a hydrogel formed from a biologically acceptable polymer material and does not degrade in aqueous environments

In another embodiment, microbeads or millicylinders loaded with one or more GFs are encapsulated within the support. The amount of GF released by the microbeads is adjusted by controlling the quantity of microbeads embedded in the support.

In some embodiments the supports can float on or just below the surface of culture media.

In other embodiments the supports are configured for removal from culture media.

In some embodiments the supports do not degrade in cell culture media or in the presence of biologic, hydrolytic or enzymatic conditions.

In a further embodiment the hydrogel support comprises a polyethylene glycol polymer.

In one embodiment, the hydrogel support is loaded with beads that carry growth factors.

In one implementation the microbeads are StemBeads®.

In a further implementation the StemBeads® are loaded with FGF.

In another embodiment, the microbeads beads contain a variety of GFs.

In a still further alternative, the beads release GFs over a period of time.

In another embodiment, the beads include magnetic particles or beads.

In a still further embodiment, a recovery device such a wire, string, thread or fishing line is attached to the hydrogel support.

In one implementation one or more feeding devices are deposited into the same cell culture.

In another implementation hydrogel supports with different GF payloads are deposited into a cell culture and then selectively removed.

These and other embodiments and implementations are described in more detail below.

### DESCRIPTION OF DRAWINGS

FIG. 1 is a schematic that depicts a feeding device comprised of degradable microbeads releasing growth factors loaded into a non-degradable hydrogel support with open lattice structure and deployed into a cell culture well containing medium.

FIG. 2A is a flow diagram describing manufacture of feeding devices from StemBeads FGF2® loaded into a 16 μL PEG hydrogel support via photochemistry.

FIG. 2B is a graph that demonstrates the amount of FGF2 released into cell culture medium over 7 days from a PEG hydrogel support loaded with StemBeads FGF2® set to release at 10 ng/mL when added into 2 mL of medium at 37° C. (n=3, error bars=st dev).

FIG. 2C is a graph that demonstrates FGF2 levels from an 8 μL sized (1-2 mm diameter disc) PEG hydrogel support loaded with about 10,000 StemBeads FGF2® when added into 1 mL of medium can achieve the same FGF2 level as a 16 μL sized (2-3 mm diameter disc) PEG hydrogel support loaded with about 20,000 StemBeads FGF2® added into 2 mL of medium. (n=4-6, unpaired t-test, ns=not significant).

FIG. 3A is a graph that demonstrates the amount of FGF2 released into the medium between 1 and 24 hours at 37° C. comparing the levels from StemBeads FGF2® delivered into culture without a support and StemBeads FGF2® in a hydrogel support normalized to the level at 24 hours (set to 1) (n=3 hydrogel supports, error bars=st dev).

FIG. 3B is a graph that demonstrates the amount of FGF2 released into the medium between 1 and 14 days at 37° C. comparing the levels from StemBeads FGF2® delivered into culture without a support and StemBeads FGF2® in a hydrogel support (error bars=st dev; n=3, unpaired t-test * p<0.05, ** p<0.005).

FIG. 4A graphs the average FGF2 levels over 7 days at 37° C. from 16 μL sized PEG hydrogel support loaded with about 20,000 StemBeads FGF2® added into 1, 2 or 3 mL of cell culture medium. (n=3, error bars=st dev).

FIG. 4B is a graph that depicts the average FGF2 levels in a culture medium over a period of 7 days at 37° C. from 16 μL sized PEG hydrogel support loaded with about 20,000 or 100,000 StemBeads FGF2® (n=3, error bars=st dev).

FIG. 5A is a graph that demonstrates EGF and FGF2 levels released in a culture medium at 37° C. from a 16 μL sized PEG hydrogel support loaded with StemBeads EGF® and StemBeads FGF2® (n=3, error bars=st dev).

FIG. 5B is a graph that depicts the FGF2 levels in a culture medium over 6 days at 37° C. as released from a 16 μL sized PEG hydrogel support loaded with magnetic beads and StemBeads FGF2® (n=2, error bars=st dev).

FIG. 5C is a graph that depicts the FGF2 levels in culture medium over 6 days at 37° C. as released from a 16 μL sized (PEG hydrogel support manufactured with microbubbles (floating hydrogel support) and loaded with StemBeads FGF2® (n=2, error bars=st dev).

FIGS. 6A-F are graphs and histograms that compare a conventional method of culturing iPSCs with daily feeds of mTESR1 medium (containing soluble FGF2) delivered into culture without a support to the improved culture method with less frequent feeds of mTESR1 medium delivered to the culture with an FGF2 feeding device.

FIG. 6A is a graphical schematic that illustrates the FGF2 levels for cultures grown with daily feeds of mTESR1 medium delivered into culture without a support (method #1).

FIG. 6B is a pie graph that illustrates the time cultures spend at different levels of FGF2 when cells are grown with daily feeds of mTESR1 medium delivered into culture without a support (method #1).

FIG. 6C is a graphical schematic that illustrates the FGF2 levels for cultures grown with less frequent feeds of mTESR1 medium delivered with an FGF2 hydrogel feeding device (method #2).

FIG. 6D is a pie graph that illustrates the time cultures spend at different levels of FGF2 when cultures are grown with less frequent feeds of mTESR1 medium delivered with an FGF2 hydrogel feeding device (method #2).

FIG. 6E are histogram plots of flow cytometry data where the percent of cells that are positive for the pluripotency marker Tra-1-60 are labeled. These plots compare three iPSC lines cultured with daily feeds of mTESR1 medium (method #1, top graphs) compared to less frequent feeds of mTESR1 medium delivered with an FGF2 hydrogel feeding device (method #2, bottom graphs).

FIG. 6F are histogram plots of flow cytometry data where the percent of cells that are positive for pluripotency marker SSEA4 are labeled. These plots compare two iPSC lines cultured with daily feeds of mTESR1 medium (method #1, top graphs) compared to less frequent feeds of mTESR1 medium delivered with an FGF2 feeding device (method #2, bottom graphs).

FIGS. 7A-C are graphs that compare 5 different methods to grow iPSCs and demonstrates improved mesoderm differentiation is achieved when iPSCs were cultured with an FGF2 feeding device.

FIG. 7A graphs the mesoderm marker brachyury gene expression of mesoderm cultures derived from iPSCs cultured with daily feeds of mTESR1 medium (containing soluble FGF2) (method #1) in comparison expression after less frequent feeds of mTESR1 medium delivered with an FGF2 hydrogel feeding device (method #2), (n=3 cell lines; n=2-3 wells per line; ** p<0.0005).

FIG. 7B graphs the mesoderm marker brachyury gene expression of mesoderm cultures derived from iPSC cultured with 3-times a week feeds of mTESR1-Plus medium (containing stabilized soluble FGF2) delivered into culture without a feeding device (method #3) in comparison to less frequent feeds of mTESR1-Plus delivered with an FGF2 feeding device (method #4), (n=3 cell lines; n=2-3 wells per line; **** p<0.00005).

FIG. 7C graphs the mesoderm marker brachyury gene expression of mesoderm cultures derived from iPSC cultured with feeds of mTESR1 medium delivered with StemBeads FGF2® (no hydrogel support, method #5) and compared to feeds of mTESR1 medium delivered with an FGF2 hydrogel feeding device (method #2), (n=3 cell lines; n=2-3 wells per line; * p<0.05).

FIGS. 8A-G are graphs that compare a conventional method of culturing iPSCs with daily feeds of E8 medium (containing soluble FGF2) to the improved culture method with less frequent feeds of E8 medium (made up without soluble FGF2) delivered to the culture with an FGF2 feeding device.

FIG. 8A is a graphical schematic that illustrates the FGF2 levels for cultures grown with daily feeds of E8 medium with soluble FGF2.

FIG. 8B is a pie graph that illustrates the time cultures spend at different levels of FGF2 when cells are grown with daily feeds of E8 medium with soluble FGF2.

FIG. 8C is a graphical schematic illustrates the FGF2 levels for cultures grown with an FGF2 feeding device added into E8 medium without soluble FGF2.

FIG. 8D is a pie graph that illustrates the time cultures spend at different levels of FGF2 when cultures grown with an FGF2 feeding device added into E8 medium without soluble FGF2.

FIG. 8E graphs the endoderm marker SOX17 gene expression of endoderm cultures derived from iPSC cultured with daily feeds of E8 medium (with soluble FGF2) compared to less frequent feeds of E8 medium (without soluble FGF2) delivered with an FGF2 hydrogel feeding device, (n=1 cell line; n=3 wells; unpaired t-test **** p<0.00005).

FIG. 8F graphs the mesoderm marker brachyury (T) gene expression of mesoderm cultures derived from iPSC cultured with daily feeds of E8 medium (with soluble FGF2) delivered into culture without a hydrogel support in comparison to less frequent feeds of E8 medium (without soluble FGF2) delivered with an FGF2 hydrogel feeding device, as disclosed herein (n=1 cell line; n=3 wells; unpaired t-test *** p<0.0005).

FIG. 8G graphs the ectoderm marker PAX6 gene expression of ectoderm cultures derived from iPSC cultured with daily feeds of E8 medium (with soluble FGF2) compared to less frequent feeds of E8 medium (without soluble FGF2) delivered with an FGF2 feeding device, (n=1 cell line; n=3 wells; unpaired t-test *** p<0.0005).

FIG. 9 is a graph that demonstrates cerebral organoids have improved levels of cortex neuronal subtypes when organoids are generated from iPSC cultured with less frequent feeds of mTESR1 medium delivered to the culture with an FGF2 feeding device compared to conventional method of daily feeds of mTESR1 medium (containing soluble FGF2) as shown by higher gene expression levels of positive cerebral cortex markers (PAX6, FOXG1, TBR1, EMX2) from 2-month cerebral organoids (n=2 cell lines; n=3 organoids pooled per line or n=3 individual organoids per line; * p<0.05 ***p<0.0005).

### DETAILED DESCRIPTION

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein the term “non-degradable” refers to biologically acceptable materials that do not break down or deteriorate chemically. More specifically the term refers to biologically acceptable plastics and other materials that do not deteriorate or break down in cell culture medium including by way of non-limiting example, the cell culture mediums disclosed herein. The definition also embraces materials that do not deteriorate or break down when exposed to biological, hydrolytic or enzymatic conditions.

As used herein the term “small molecules” refers to those compounds with a molecular weight below 1000 Daltons.

As used herein, the term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

As used herein the term “biologically acceptable” means the material that is generally safe, non-toxic, and neither biologically nor otherwise undesirable and includes that which is acceptable for veterinary use as well as human pharmaceutical use.

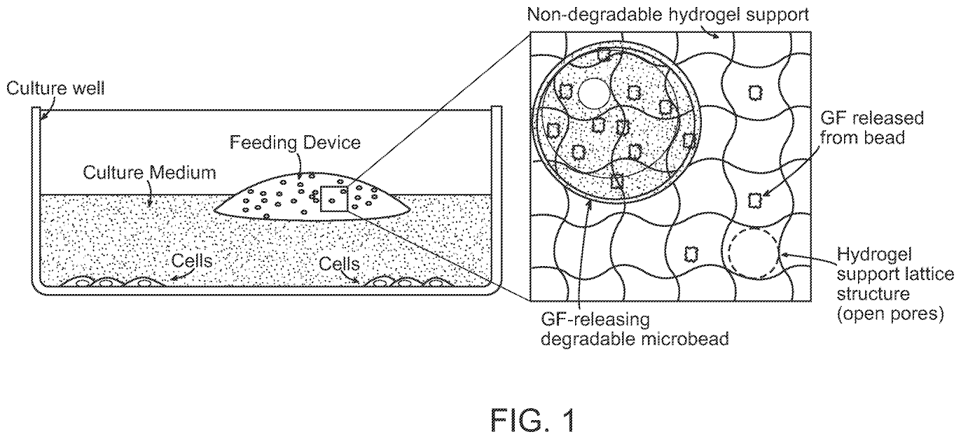

The feeding devices disclosed herein generally comprise a hydrogel support that is a non-degradable, biologically acceptable, inert material that can hold a cargo or payload, such as for example degradable microbeads. The hydrogel support material has an open lattice structure that allows GFs to diffuse through and be released into the cell culture medium but is small enough to retain the microbead cargo (See FIG. 1).

The inert non-degradable hydrogel support material prevents the feeding device from interfering with growth of cells in culture. This support can be easily added and removed from cultures. The hydrogel support can comprise a variety of different polymers including by way of non-limiting example synthetic polymers (e.g. polyethylene glycols, polyacrylamides) and naturally occurring polymers (e.g. polysaccharides, polypeptides).

The hydrogel support may contain a cargo, such as multiple types of GF releasing microbeads, colored beads, magnetic beads, air bubbles and/or a tether (which can be for example a wire, filament, thread or string), to assist in its functionality as a removable feeding device for cell culture. Using one embodiment of the feeding devices disclosed herein it has been shown that cell culture quality is significantly and surprisingly improved as compared to conventional feeding methods (soluble GF added daily without a hydrogel support) and feeding methods using microbeads without a hydrogel support.

Unlike other degradable materials used to deliver factors to cell cultures, the hydrogel supports described herein do not degrade in cell culture media or in the presence of biologic, hydrolytic or enzymatic conditions. The devices disclosed herein are also ‘inert’ defined as having anti-fouling properties by discouraging non-specific protein adsorption via highly hydrophilic hydrogel polymer backbone. ‘Inert’ is also defined as a material that does not contain cell binding motifs and does not promote cell attachment. These non-degradable and inert properties of the hydrogel support are beneficial to the feeding device as they prevent the device from interfering with the cells in culture. Previous technology, such as degradable GF microbeads comprised of PLGA or naturally occurring polymers constructs (collagen, gelatin, laminin, fibrin, matrigel, etc.) are not inert to cells and have been shown to incorporate into cell monolayers and 3D organoids. Additionally, these materials are degradable and thus can release byproducts that can alter the cell culture environment. The removable feeding devices described herein circumvent these concerns. For example, before the feeding devices disclosed herein, degradable microbeads (e.g. StemBeads®, StemCultures LLC) added into a 2D cell culture would stick to cells, multiple washes were required to assist in the removal of microbeads and a full removal was not readily achieved. StemBeads® are controlled release micro particles composed of a biodegradable polymer that is loaded with one or more GFs such as recombinant FGF2 (StemBeads® and are available from StemCultures, 1 Discovery Drive, Rensselaer N.Y.) (See for example U.S. Pat. No. 8,481,308 incorporated herein in its entirety by reference).

With the devices disclosed herein, the degradable microbeads (e.g. StemBeads®, StemCultures LLC) are loaded into an inert non-degradable hydrogel support, the microbeads are retained within the feeding device, do not intermingle with the cultured cells and full removal of the device and its bead cargo is easily achieved without any washing steps.

The hydrogel supports described herein are preferably transparent and do not interfere with imaging of the cell cultures. The cargo carried by the support, such as beads, or particles may not be transparent. The hydrogel devices can be added to and later removed easily from cell cultures, achieving a controlled environment and essentially complete and efficient removal of GFs from the culture, with negligible (not enough to provide detectable bioactivity) GF remaining after removal of the device bearing the GF from the culture. Removal of the feeding device from the cell culture does not require a medium exchange (i.e. cell culture media) which is required to remove residual degradable additives. This generates savings on culture media and labor while providing controlled growth signaling to cells.

### Hydrogel Support:

The supports disclosed herein are primarily made from hydrogels. Hydrogels are water insoluble, cross-linked three dimensional polymeric networks, which have the ability to hold water within the spaces available among the polymeric chains. Crosslinking facilitates insolubility in water and provides required mechanical strength and physical integrity. Hydrogel is mostly water (the mass fraction of water is much greater than that of polymer). The ability of a hydrogel to hold significant amounts of water implies that the polymer chains must have at least moderate hydrophilic character. Like a liquid, small molecules diffuse through a hydrogel.

The water holding capacity of the hydrogels arise mainly from the presence of hydrophilic groups (e.g., amino, carboxyl and hydroxyl groups), in the polymer chains. The greater the number of hydrophilic groups, the greater the water holding capacity, while with an increase in the cross-linking density there is a decrease in the equilibrium swelling. Hydrogels are cross-linked polymeric networks and these networks provide the hydrogel with a three-dimensional polymeric structure.

Polymers useful for making the hydrogel feeding devices disclosed herein are those that are inert, non-degradable and form sufficiently open lattice structures to allow small molecules/proteins to diffuse through but that also retain bead components within their matrix. The hydrogel supports open lattice structure can have a pore size between about 20 nm to about 10 μm but are preferably in the range between 500 nm and 5 μm.

### Hydrogel Support Polymers

A wide range of biologically acceptable polymers that exist as hydrogels including synthetic polymers (e.g. polyethylene glycols, polyacrylamides) and naturally occurring polymers (e.g. polysaccharides, polypeptides) can be used to prepare the supports described herein.

In one preferred embodiment PEG-diacrylate monomers (cat #ACRL-PEG-ACRL-20K-5g, Laysan Bio Arab, AL; cat #ACLT-PEG-ACLT, JenKem Plano, Tex.) are used for the hydrogel support. Alternatively, other hydrogel forming polymers include acrylate functionalized polysaccharides such as alginate (cat #5310, Advanced Biomatrix Carlsbad, Calif. 92010; PhotoAlginate-INK, CELLINK Boston, Mass.; cat #912387, Sigma-Aldrich St. Louis Mo.) and hyaluronic acid (cat #5212, Advanced Biomatrix Carlsbad, Calif.; cat #D16110025376, CELLINK Boston, Mass.; cat #HA40K-1, LifeCore, Chaska, Minn.) and Poly (2-hydroxyethylmethacrylate) (pHEMA) (cat #529265-5G, Sigma-Aldrich, St. Louis Mo.). Also, polyacrylamide (cat #9003-05-9, Sigma-Aldrich, St. Louis Mo.; cat #1610154, Bio-Rad, Hercules, Calif.) hydrogels can also be used as a hydrogel support.

In preferred embodiments, the support is a hydrogel made from a polyethylene glycol (PEG) polymer. PEG is an FDA approved material with excellent non-toxic, anti-biofouling, non-immunogenic properties due to its flexible and hydrophilic polymer chains. PEG can be functionalized and cross-linked to form a hydrogel. In one example, the hydrogel support can be comprised multi-armed PEGS (i.e. 8-arm PEG-norbornene (8ARM(TP)-NB, JenKem, Plano, Tex.) or 4-arm PEG-maleimide (4ARM-MAL, JenKemPlano, Tex.; 4arm-PEG-MAL-20K-1g, Laysan Bio Arab, AL)) with PEG-dithiol crosslinks (SH-PEG-SH-3400-5g, Laysan Bio, Arab, AL) via chain growth polymerization (i.e. thiol-ene chemistry).

In one preferred embodiment, PEG monomer functionalized with acrylate groups (preferred) is used to make a hydrogel support by crosslinking of the monomers via chain growth polymerization chemistry. For example, polyethylene glycol diacrylate (PEGDA) (cat #ACRL-PEG-ACRL-20K-5g, Laysan Bio Arab, AL; cat #ACLT-PEG20K-ACLT, JenKem Plano, Tex.) is added to an aqueous solution (e.g. water or phosphate buffered saline (PBS)) and mixed with the desired cargo (e.g. microbeads). In one embodiment the molecular weight (MW) of the PEGDA monomer is 20 KDa but in other embodiments PEGDA monomers having a 1\4W between 1 KDa and 200 KDa and preferably between 15 KDa and 35 KDa may be used to create the support. In one embodiment the final PEG concentration in the precursor PBS or water solution prior to polymerization of the hydrogel support is 0.1 g/mL (10% weight by volume) and in other embodiments it can be between about 0.05 g/mL (5% weight by volume) and 0.4 g/mL (40% weight by volume).

### Hydrogel Support Polymerization

One preferred way to polymerize chemically cross-linked hydrogels is by using actinic light exposure and a photo-initiator to initiate the reaction between acrylate functionalized monomers to form a cross-linked hydrogel e.g. methacrylate alginate, methacrylate hyaluronic acid, PEG-diacrylate hydrogel.

One preferred photo-initiator used is P-Phenyl-P-(2,4,6-trimethylbenzoyl) phosphinic acid (LAP), available from companies such as Tocris Bio-Techne (Minneapolis Minn., cat #6146) and Advanced Biomatrix (Carlsbad, Calif. cat #5269). The final LAP concentration in PEG solution is 10 mM. In other embodiments LAP concentration can be used between 1 μM to 100 mM, more preferably between 1 mM and 20 mM concentration to initiate photopolymerization. Other photo-initiators that are useful in preparing the hydrogel supports disclosed herein include Irgacure-2959 (cat #410896, Sigma-Aldrich, St. Louis Mo.; cat #5200, Advanced BioMatrix, Carlsbad, Calif.) 2,2-dimethoxy-2-phenylacetophenone (cat #24650-42-8, Sigma-Aldrich St. Louis Mo.), eosin Y (cat #15086-94-9, Sigma-Aldrich St. Louis Mo.) and Ruthenium (cat #5248, Advanced BioMatrix, Carlsbad, Calif.).

To activate the polymerization for polymer solutions containing LAP as the photo-initiator, the solutions are exposed to UV light (390 nm wavelength, between 365-400 nm) for 30 seconds. UV exposure time can be between about 5 seconds and about 5 minute to polymerize the droplet based on UV power, droplet size, photo-initiator type and concentration. The UV light wavelength parameters (i.e. wavelength, strength, exposure time) will all be selected based on the photo-initiator type and concentration being used. Other photo-initiators that are useful in creating the devices disclosed herein and which require UV light (wavelength ˜365 nm) for activation include Irgacure-2959 and 2,2-dimethoxy-2-phenylacetophenone. Photo-initiators that require visible light (wavelength ˜510 nm) are eosin Y and Ruthenium and require exposure time between 1 minute and 1 hour.

### Hydrogel Support Sizes and Shapes

The hydrogel monomer solution (prior to cross-linking) is mixed together with a desired cargo (e.g. microbeads loaded with one or more GFs) to uniformly disperse cargo in the solution. The hydrogel supports disclosed herein can be made into different sizes. Prior to cross-linking the monomer/microbead liquid mixture can be formed into different geometric shapes and sizes. Thus, the mixture can be deposited into shaped receptacles that may be in the form of generally circular droplets (size between 1 and 20 mm in diameter), balls, squares, rectangles, triangles or free form shapes. Changing the volume of the droplet pipetted from precursor hydrogel-cargo solution can provide different circular-shaped discs with sizes such as 0.5 mm, 1 mm, 2 mm, and 5 mm in diameter.

The minimum and maximum size of devices has no theoretical limit beyond the smallest size needed to encapsulate the desired number and size of beads, which can be nano- or micron sized and the largest size needed for the specific application, such as compatibility with a large bioreactor. Preferred volumes of the hydrogel feeding devices are between 1 μL and 1000 μL.

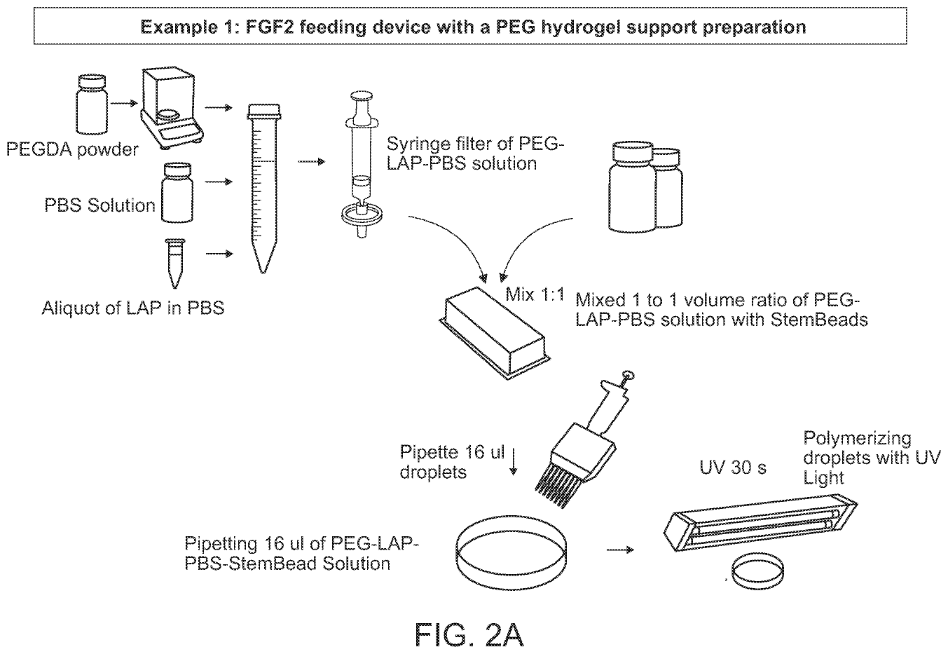

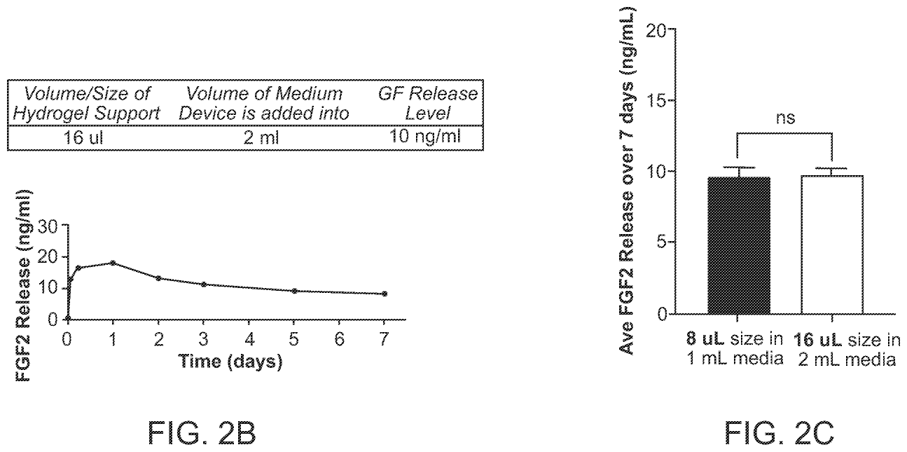

In a non-limiting example, droplets are pipetted on a hydrophobic surface, such as a non-tissue treated plastic dish to form disc shaped support devices. In one embodiment, small volumes (e.g. 16 μL) of the monomer/bead mixture are pipetted to form circular feeding supports about 2-3 mm in diameter and 0.5-1 mm in center thickness and then exposed to actinic light to crosslink the monomer and form a hydrogel (see FIG. 2A). These dimensions are the size of the devices at manufacture; however, the finished hydrogel product can swell to between about two to three times its initial size when added to a solution e.g. a culture medium.

The preferred device volume for a 6-well or 12-well culture dish is between about 10-20 μL and 1-4 mm in diameter (prior to swelling). The preferred device volume for a 24-well or 48-well culture dish is between 5 and 10 μL and 0.5-1 mm in diameter (prior to swelling). Feeding devices can be made to release the same level of GF and be packaged in different sized hydrogel supports for different culture vessel sizes (i.e. different medium volumes).

To make different shaped versions of the removable device, a photomask can be used with the UV light to photo crosslink specific shapes such as squares, rectangles, triangles, donuts, rods, etc. Another way to generate different shaped devices is to bio-print with a 3D printer (e.g. Bio X, D16110020717. CELLINK, Boston, Mass.). Devices with different shapes can be generated by extruding precursor solution from a flow-controlled nozzle into a pre-designed shape or pattern and then subsequently crosslinking with a UV light source. This will create a polymer support of the pre-designed shape or pattern. In another implementation, microbeads can be spatially controlled within the three-dimensional hydrogel structure and then the hydrogel support cross-linked to lock the microbeads into position. This can be accomplished by bio-printing different precursor solutions containing different amounts of cargo, i.e. StemBeads® in a pre-designed pattern. This can then provide a gradient or pattern of release relative to the cells in culture.

### Hydrogel Support Porosity and GF Release Kinetics

Hydrogel support characteristics can alter the GF release kinetics by adjusting the lattice structure through manipulation of molecular weight of the monomer, crosslinking densities and/or monomer concentration.

For example, hydrogel supports made up of lower molecular weight polymer monomers (i.e. PEGDA monomers with MW between 1 to 10 KDa) will facilitate slower GF release and slower diffusion rates compared to hydrogel supports comprised of monomers with higher MW (i.e. PEGDA monomers with MW between 10 KDa-100 KDa).

Similarly, hydrogel supports comprised of high crosslink densities will facilitate slower GF release (i.e. slower diffusion rates) than hydrogel supports made up of fewer crosslinks. In one non-limiting example, increasing cross link density is accomplished by decreasing the reaction time of free radical polymerization (i.e. reducing exposure time of PEGDA monomers to UV light). In another example, instead of using a 4-arm PEG monomer in a step-growth polymerized hydrogel support, the crosslink density is increased by using an 8-arm PEG monomer in the hydrogel support.

Adjusting the monomer concentration of the hydrogel support is another approach to alter the rate of GF release. In a preferred embodiment, the hydrogel support is comprised of 20% w/v PEGDA monomers and the GF release is retarded by increasing amount of PEGDA monomers from 20% to 40%. In the same vein, the rate of GF release can be increased by reducing the quantity of PEGDA monomers used to create the hydrogel support.

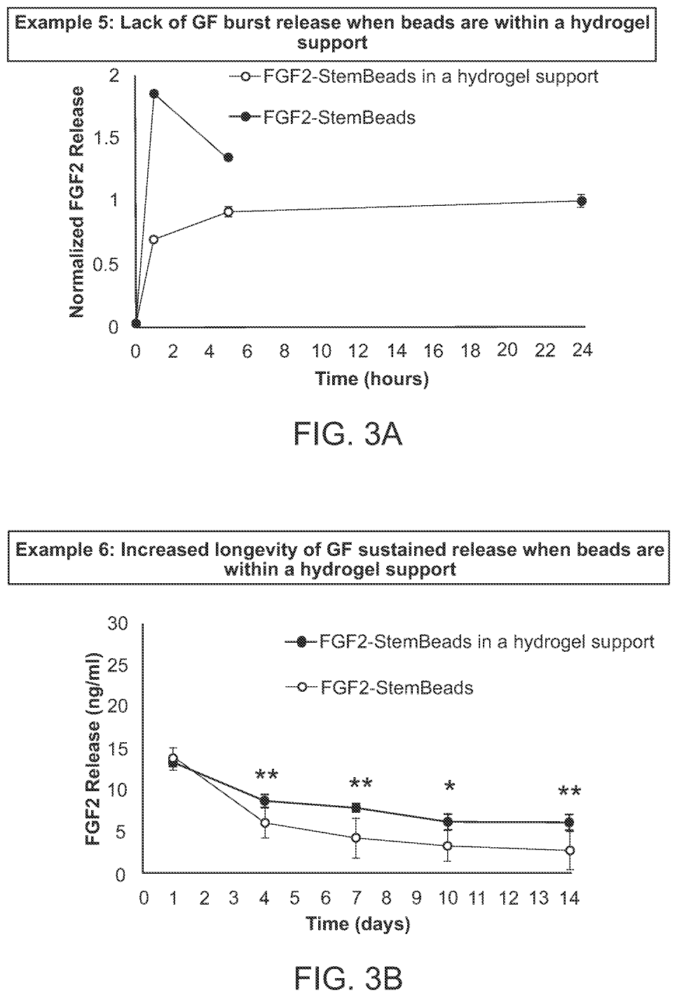

Hydrogel supports provide a method to avoid burst effects of microbeads. The burst effect is an undesired event in controlled release technologies but an often-unavoidable outcome. The burst effect is defined as a short burst of high concentrations of GF released after the initial exposure to the solution. Burst effects can occur when large concentration gradients exist between the microbeads and the medium. When microbeads are first added to the medium, high GF concentrations are localized within the microbead (e.g. 1000 ng/mL) and there is no/low GF in the medium (e.g. 0-10 ng/mL). Burst effects can also occur when some of the GFs are located on the surface of the microbead. The slower diffusion rate that exists through the hydrogel support provides a localized microenvironment around the beads to dampen this gradient and can reduce and/or avoid the burst effect (See FIG. 3A).

Hydrogel supports provide a method to extend the controlled release of GFs. Lower molecular weight, higher concentration of monomers and/or higher degree of crosslinking will result in smaller pore lattice structure, thus decreasing the rate of diffusion through the hydrogel support and can contribute to the control over GF release by extending, delaying and/or slowing the GF release rates. The slower diffusion rate through the hydrogel support provides a localized microenvironment around the beads to slow the degradation of the beads and extend the sustained release time period (See FIG. 3B).

### Dehydrated Hydrogel Supports for Storage and Handling

The non-degradable hydrogel support described herein, is capable of swelling or de-swelling reversibly in water and retaining large volumes of liquid in the swollen state. In a preferred embodiment, feeding devices are dried and dehydrated after manufacture for storage and ease of handling. The drying and dehydration process removes essentially all of the water from the hydrogel composition. After polymerization reaction, the support is transparent. However after drying the support is no longer transparent but is dry to the touch. When hydrogel support is dehydrated, shelf life of hydrolytic degradable cargo (i.e. PLGA microbeads) can be extended. The polymerized hydrogels are dried for between about 12 to 24 hours at a temperature between about 18° C. and about 22° C. and preferably at about 20° C. The preferred humidity for drying is between 30 and 50%, and is preferably about 40%. The hydrogel device is dried to remove liquid and stored in this dried format in an airtight container at −20° C. or refrigerated at 4° C. The hydrogel device can also be stored in solution as a wet format at 4° C. Furthermore, the hydrogel support has improved handling characteristics, i.e. easier to pick up with forceps, in the dehydrated format. Once added to medium, the hydrogel support will rehydrate and can swell two to three times in size. The GF begins to be released from the microbeads encapsulated in the hydrogel support when hydrated.

### Microbeads Description:

Various types, amounts and combinations of microbeads can be loaded into a hydrogel support to achieve different device embodiments such as GF-releasing, small molecule-releasing, endotoxin-removing and cellular output measuring devices. Microbeads can be nanometers to microns in diameter (e.g. generally between about 0.01 μm to about 1 mm in size). One preferred microbead for use in the devices disclosed herein is between about 10-100 μm in diameter. Microbeads preferred for use in the devices disclosed herein are available from various vendors including from StemCultures LLC Rensselaer N.Y., Miltenyi Biotec Gaithersburg, Md., Cube Biotech Wayne Pa., Cospheric Santa Barbara, Calif., etc.

While microbeads are customarily ball shaped, the microbeads useful in the hydrogel feeding devices disclosed herein can be of any geometric shape. Thus, the microbeads may for example, have a ball shape, or be configured in the shape of a pyramid, brick or cube. This includes different forms of particles including solid, hollow, amorphous, and solubilized. Microbeads useful in the hydrogel feeding devices disclosed herein are preferably PLGA microspheres but can be of other degradable biocompatible plastics such as poly (lactic acid), poly (glycolic acid), poly (e-caprolactone). The microbeads can also be made of non-degradable inorganic materials such silica or non-degradable petrochemical plastics such as polypropylene and polystyrene. The microbeads can also be made of naturally occurring materials such as alginate, collagen, gelatin, hyaluronic acid, chitosan, fibrin, and agarose. The microbeads may be magnetic beads (e.g. made of iron oxide particles such as magnetite) or hollow beads. Microbeads useful in the hydrogel supports disclosed herein include, by way of non-limiting example controlled-release degradable (GF) beads (e.g. StemBeads® available from Stem Cultures LLC Rensselaer, N.Y. 12144—see U.S. Pat. No. 8,481,308), agarose magnetic beads (cat #130-093-657, Cube Biotech Wayne, Pa.), polyethylene-colored microspheres (cat #BLPMS-1.0027-32 um-1g, Cospheric Santa Barbara, Calif.), glass hollow microspheres (cat #HGMS-0.6 5-30 um, Cospheric Santa Barbara, Calif.) or endotoxin-removal beads (e.g. from cat #130-093-657, Miltenyi Biotec Gaithersburg, Md.). One preferred microbead implementation comprises GF encapsulated PLGA beads (StemBeads®) that have diameters in the 1-100 μm (e.g. StemBeads®).

### Growth Factors and Small Molecules:

Growth factors and small molecules, like biologic growth factors such as FGF2 or EGF, are labile in culture medium and have short half-lives. When molecules are encapsulated into microbeads, molecules are protected from aqueous environment and thus stabilized. Degradable microbeads then slowly release molecules over time and overcome these limitations.

The microbeads useful in the feeding devices disclosed herein can be loaded with growth factors such as for example FGF2, EGF, BDNF, GDNF, TGFb1, BMP4, IL2, IL34 and other cell culture media additives such fetal bovine serum (FBS). Typical GF concentrations of PLGA microbeads for cell culture range from about 0.1 to about 300 ng/mL, preferably from about 0.5 to about 20 ng/mL. The microbeads are embedded in the polymer support device.

Microbeads may be loaded with small molecule substances (molecular weight between about 300 g/mol and about 1 kg/mol) such as chir99021 (cat #4423, Tocris Bio-Techne Minneapolis, Minn.), LDN 193189 (cat #52618, Selleckchem Houston, Tex.), Dorsomorphin (cat #3093, Tocris Bio-Techne Minneapolis, Minn.), XAV 939 (cat #3748, Tocris Bio-Techne Minneapolis, Minn.). Typical small molecule concentrations in the microbeads can range between about 0.1 nM to about 100 uM and more preferably from about 50 nM to about 20 μM.

The feeding devices disclosed herein ensure the presence in a culture of a controlled concentration range of growth factor over time (e.g., at least one day, at least 2 days, at least 3 days, at least 4 days, at least 5 days, at least 6 days, at least 7 days, or longer). Thus, in preferred embodiments, one, or two, or three, or more growth factors may be delivered using the hydrogel polymer support in controlled release formulations to a cell culture at the beginning of the culturing process, and no further medium changes are required during an extended time period (e.g., for multiple days) and no additional exchanges of feeding devices are required during an extended time period (e.g., for up to 7 days or more). When loaded with degradable GF microbeads, the inert devices can release GF proteins at a relatively uniform release rate for up to 7 days or more (see FIG. 2B), enabling much less frequent medium exchanges.

In one embodiment the PEGDA monomers (20 KDa) are dissolved in aqueous solution at a concentration of 0.2 g/mL (20% weight by volume) with a water-soluble photo initiator LAP (Torcis) at 20 mM in PEGDA solution. The monomer solution is filtered for sterility (0.22 μm syringe filter) and then mixed with sterile microbeads at a 1:1 volume ratio. This final solution contains 0.1 g/mL (10% weight by volume) of PEGDA and 10 mM of LAP. The concentration of stock microbeads is determined by 1) the desired level of GF released 2) the desired volume of medium the device will be added to and 3) the specific size (volume) of each device. For example, in iPSC cultures, 8 μL FGF2 feeding devices were made to release at 10 ng/mL when added to 2 mL of medium for smaller well-plate format (i.e. 48 well plate) (See FIG. 2C). For larger well-plates (i.e. 6-well or 12-well format), 16 μL volume FGF2 feeding devices were made to release at 10 ng/mL when added to 2 mL of medium (See FIG. 2C).

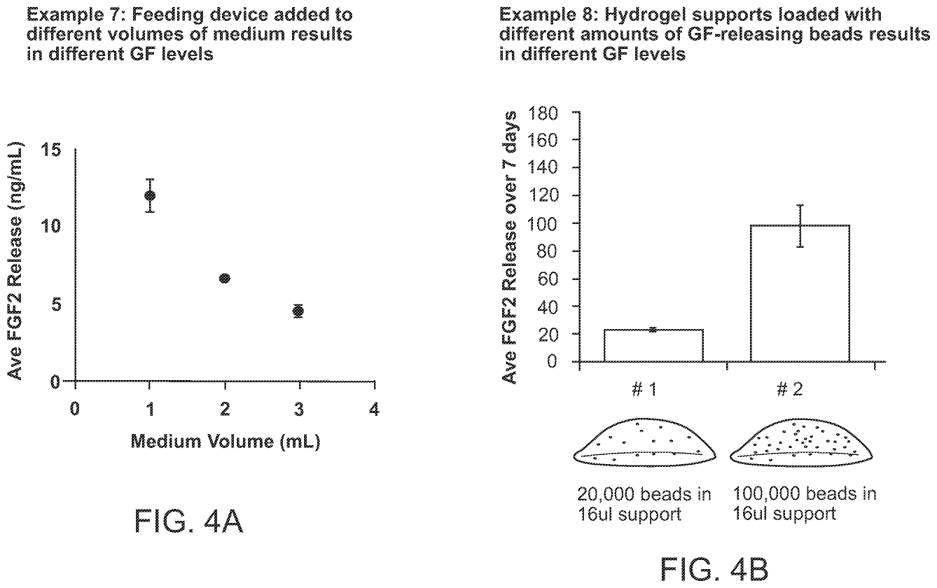

The volume of medium into which the feeding device is dispensed affects the GF concentration level. In one example, FGF2 feeding device made to release at about 12 ng/mL when added to 1 mL of medium can alternatively be added into 2 mL of medium to achieve a release level of 6 ng/mL or added to 3 mL of medium to achieve a release level of about 3 ng/mL (See FIG. 4A).

The quantity of microbeads deployed in the hydrogel support also determines the level of GF that is dispensed by the feeding device when it is installed in the culture medium. Incorporating more microbeads results in higher levels of GF being released into the culture medium. If the polymer support contains fewer beads, a lower level of GF is released into the medium. In one example, a 16 μL sized hydrogel support can be loaded with about 20,000 StemBeads FGF2® and release 20 ng/mL of FGF2 when added to 1 mL of medium at 37° C. Alternatively, a 16 μL sized hydrogel support can be loaded with about 100,000 microbeads and release 100 ng/mL of FGF2 (See FIG. 4B). This demonstrates that by using a feeding device described herein, it is possible to achieve a wide range of GF levels in the culture medium by adding only one single item alone (a hydrogel support loaded with microbeads) into a culture vessel compared to dispensing microbeads (or other degradable GF-releasing technologies) without a support which require large numbers (e.g. 20,000-100,000 StemBeads FGF2®) of beads (i.e. microbeads) to be dispensed into a culture vessel.

### Multiple Types of GFs Released Feeding Devices:

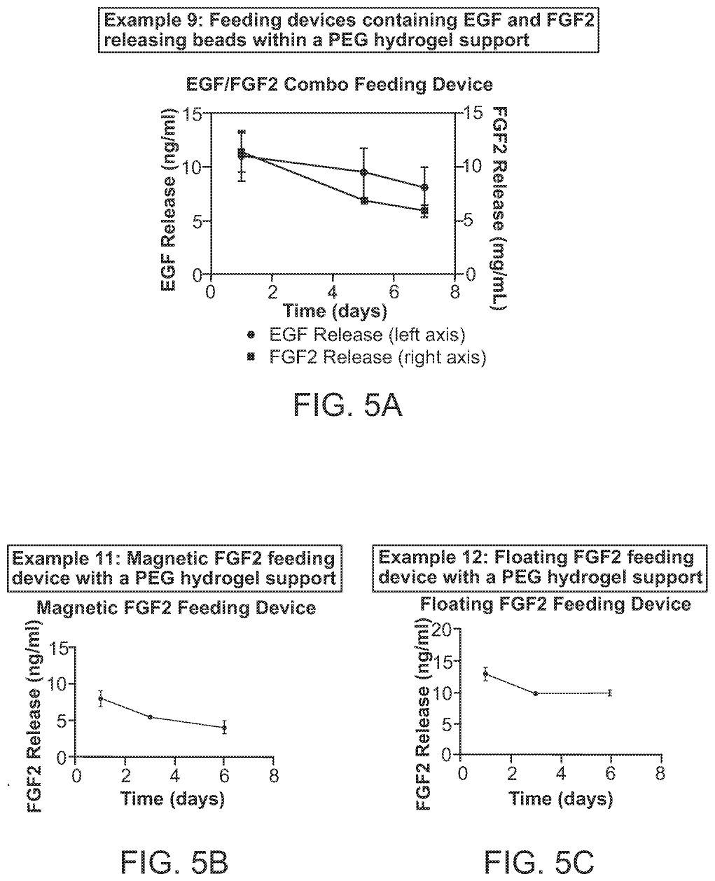

A single removable device can be loaded with different types of microbeads to perform multiple tasks at once, such as releasing different types of GFs simultaneously. For example, multiple bead types loaded with different growth factor payloads can be encapsulated into a single hydrogel support and this feeding device thus releases multiple GF types at once (See FIG. 5A). When more than one GF is loaded into a hydrogel support, this feeding device can replace complex cell culture reagents such as fetal bovine serum (FBS). Alternatively, several removable hydrogel devices can each be loaded into a single culture and controlled independently of each other.

In another implementation, the hydrogel support can be loaded with beads that release GFs at different times. This will allow one feeding device to change the GFs in the medium without a medium change. For example, one hydrogel support may have one type of microbead (e.g. silica microbeads loaded with small molecule chir99021) that releases all of its content within two days and another type of bead (delay-release double layered PLGA microbeads loaded with VEGF) in which the content release is delayed for two days after the support is installed in the culture medium.

In another implementation, the hydrogel support can be loaded with microbeads that contain living cells such as for example astrocytes or neurons. Cells secrete many GFs and this combination of GFs can be used to grow cells, differentiate cells or maintain a cell fate. In this implementation, one type of cell such as astrocytes are encapsulated into microbeads, e.g. collagen microbeads. These beads are installed within a hydrogel support. The hydrogel support will allow the encapsulated cells to secrete GF into the medium but prevent the encapsulated cells from directly interacting or co-mingling with the cells in culture. The hydrogel support allows these cells to be kept separate from the cells in culture. The cells are also easily added to or removed from the culture.

### Colored Feeding Devices:

The hydrogel supports disclosed herein may be constructed with a color. Different colored supports may carry different GFs. This enables one GF to be removed from a culture without removing any other GFs.

Color may be added into the supports by embedding colored microbeads in the support structure. Colored beds are embedded into the support as an identifying mechanism. Supports bearing a particular GF can be identified by colored beads in the support. For example, a support loaded with FGF-2 into which blue colored beads are embedded. Thus, making it relatively straightforward to identify supports bearing FGF2. Different color microbeads can be embedded in supports bearing different GFs. Hydrogel supports with different GF payloads can be added to the cell culture and then removed selectively. These devices loaded respectively with FGF2-containing beads (and for example red color microbeads) and Fetal Bovine Serum (FBS) containing beads in a support that also contains blue color microbeads can be placed into a cell culture receptacle, then the device loaded with FGF2-containing beads can be readily identified and then removed leaving the FBS-containing device in the culture. This cannot be achieved using biodegradable additives such as FGF2-beads and FBS-beads because once they are added to the culture they mix and cannot be readily separated or identified.

In such an arrangement, upon removal of the support, each GF is completely removed from the culture medium leaving negligible (not enough to provide a detectable bioactivity) levels of that GF in the culture, by simply removing the support bearing the GF from the culture. The color incorporated into the hydrogel supports makes it possible to have multiple feeding device of different colors, each bearing a different GF or combination of GFs. For example, a dye can be incorporated in the hydrogel composition. In one implementation StemBeads® containing FGF2 are embedded in a hydrogel support tinted with a blue dye (and any other GFs or small molecules are embedded in hydrogel devices each having a different color), the FGF2 is removed from the culture by simply removing the blue hydrogel device with a sterile forceps or via aspiration. Easy removal of the devices containing these small molecules and/or growth factors enables such a sequence without having to change the cell culture medium.

### Magnetic Feeding Devices:

The removable feeding devices described herein can be magnetized to facilitate easier removal from cultures, for example in large suspension cultures and allowing devices to be controlled (add/removed/moved/anchored/float) using magnetic force (See FIG. 5B).

In this implementation, magnetic particles (e.g. iron, steel, nickel, cobalt, gadolinium, Neodymium) are incorporated into the hydrogel structure to create e.g. a feeding disc that can be retrieved with a magnet. In one implementation, magnetic beads are added into a precursor solution prior to hydrogel photo-crosslinking. A magnet is used to remove the hydrogel device containing the magnetic beads from large suspension cultures, such as a spinner flask or bag. In addition, an external magnet is employed to control the precise location of the magnetized device within a culture, such as positioning the device on one side of the culture flask or floating near the surface of the medium. The hydrogel feeding device can also double as a stir bar when it is made into a rod shape and the culture flask is placed on a stir plate.

### Floating Feeding Devices:

In another feature, the feeding devices are manufactured to float to assist in easier removal and prevent device interactions with the cells growing at the bottom of the dish. In this implementation, gas (e.g. air, oxygen, nitrogen) bubbles are introduced into a hydrogel precursor solution prior to hydrogel photo-crosslinking. In a non-limiting example, bubbles were added to the hydrogel-microbead precursor solution prior to polymerization (e.g. prior to exposure to actinic light) by triturating (i.e. pipetting up and down several times) with a 200 μL pipette tip that did not contain liquid (i.e. contained air) and thus the titration introduced air bubbles into the solution. The bubbles were maintained in the hydrogel support after the hydrogel was polymerized (i.e. exposed to actinic light). This resulted in feeding devices that released GFs and floated on or just below the surface of the culture medium (See FIG. 5C).

Feeding Devices with a Tether Attachment:

In another implementation, a hydrogel support can encapsulate a tether such as for example one end of a wire, suture, fluorocarbon filament or nylon thread, to act as a mechanism for retrieval of the feeding device from a cell culture. In one implementation, during manufacture of the support the precursor hydrogel solution is pipetted on top of one end of the tether prior to photo-crosslinking. In one embodiment, a float (hydrogel support loaded with bubbles) is attached at the opposite end of the tether from the hydrogel support containing GF releasing microbeads. During crosslinking the tether will be encapsulated into the hydrogel, linking the two together. The float support can be used to remove the GF releasing feeding device from the culture medium. In another embodiment, tethers can help hold multiple feeding devices together and assist in the addition or removal from a culture.

### Devices to Remove Unwanted Factors:

Cell culture hydrogel feeding devices described herein can also help to remove unwanted factors from media. For example, endotoxins are lipopolysaccharides (LPS) that can be left over when biological materials, such as recombinant proteins or plasmids are produced in E. coli bacteria. Even small levels of endotoxins introduced into mammalian cells cultures can be significantly harmful. As a safety feature, endotoxin-removal devices can be added directly to cultures to remove any residual LPS. These are made by adding endotoxin-removal beads (cat #ab239707, Abcam 1 Kendall Sq Ste B2304 Cambridge, Mass. 02139 United States; cat #130-093-657 Miltenyi 201 Clopper Rd, Gaithersburg, Md. 2087) into precursor hydrogel solutions, pipetting the droplets and performing UV crosslinking, as described above. These hydrogel devices have encapsulated endotoxin-removal beads. When these devices are added into culture vessels, LPS will bind to the beads within the device and then be isolated from the culture medium and safely removed.

Antibodies can be included in the support to bind to and remove molecules from the cell culture media. For example, microbeads bearing tau antibodies can be loaded onto a support in order to bind soluble tau present in cell culture medium. Cells secrete tau and a build-up of tau protein in culture media can be toxic. Thus this device can help to maintain cell cultures in a healthy state by collecting soluble tau and keeping this toxic protein from interacting with the cells (2D) or brain organoids (3D) in the culture.

### Devices to Measure Cellular Outputs:

Removable devices disclosed herein can be used to measure cellular outputs. For example, pH sensitive dyes such as phenol red and metabolic dyes such as Alamar Blue (Thermo Fisher) can be localized within microbeads and encapsulated into removable devices. Changes in the color of these dyes would reflect changes in the culture without the dyes or similar reporters directly interacting with the cells themselves. In one example, phenol red is chemically conjugated into the hydrogel support. This device is added into phenol-free medium. When the pH changes in the culture, the feeding device changes color from red to orange. This is useful for cultures in which phenol red interferes with the cells or imaging assay. The device can still report pH changes in real time without disruption of the cells in culture/other readouts that require phenol-free medium. In another embodiment, the device can include engineered fluorescent reporter cells such as cells loaded with a calcium indicator dye to assess intracellular calcium levels or cells loaded with a pH sensitive dye to examine lysosomal function are encapsulated within the hydrogel. The reporter cells fluoresce after exposure to a composition in the medium and are used to read the level of cellular products released into the medium. Such devices are readily removed for further readout quantifications or downstream cell applications without a medium change.

Devices to Add Reagents into the Medium (not Sustained Release):

Hydrogel supports can be manufactured with solutions of cell culture reagents such as buffers (i.e. HEPES, sodium bicarbonate) and lipids (i.e. cholesterol, oleic acid) that are to be released into the medium immediately (not sustained release). This is useful for culture medium components that are not labile. In one embodiment, PEG hydrogel supports contain 1 g of PEGDA monomers (20 KDa MW) that is dissolved into 1 mL of HEPES buffer (1 M). PEG monomers are polymerized and HEPES buffer is encapsulated into hydrogel. When the hydrogel support is added into cell culture medium, HEPES buffer is released into the medium within the first 30 minutes to achieve a HEPES concentration of 10 mM to help maintain the desired pH in the cell culture medium.

### Utility of Feeding Devices for Cell Culture:

Feeding devices can be used in three different stages of cell culture. Feeding devices can be used to help (1) the growth of cells, such as replacing FBS, (2) to maintain a desired cell fate, such as iPSCs, NPCs etc. or (3) to differentiate cells from a progenitor cell into a desired cell fate. For example, a sequence of different small molecules and or biologic growth factors such as FGF2 are applied over days-to-weeks to a stem cell culture to obtain a desired stem cell product. Sustained levels of FGF2 supplied by an FGF2 feeding device to iPSC cultures are able to maintain pluripotency across iPSC lines better then cultures fed by conventional methods (i.e. feeding daily with high levels of soluble FGF2 with no support), FGF2 feeding devices improve direct differentiation of iPSCs into endoderm, mesoderm and ectoderm progenitor cells compared to alternative feeding methods including feeding with soluble FGF2, stabilized FGF2 and FGF2-releasing microbeads (i.e. StemBeads FGF2®) FGF2 feeding devices used to culture iPSCs improve organoid production compared to conventional iPSC culture method (feeding daily with high levels of soluble FGF2).

The following examples further illustrate the manufacture and use of the feeding devices disclosed herein.

### EXAMPLES

### Example 1: FGF2 Feeding Device with a PEG Hydrogel Support Preparation

This example describes manufacture of FGF2 feeding devices using PEG-hydrogel supports containing StemBeads FGF2® (see FIG. 2A). The loaded hydrogel support was made from a 16 μL sized droplet which yielded disc shaped devices of about 2-3 mm in diameter (before swelling) The feeding devices had a relatively uniform controlled release rate of 10 ng/mL FGF2 over 7 days when added into 2 mL of medium (see FIG. 2B).

Lyophilized recombinant FGF2 proteins were encapsulated into PLGA microbeads via double emulsion process. 5 mg of human FGF2 (Shenandoah, Warminster, Pa.) was dissolved into a 50 mL solution containing 0.6 mg/mL of magnesium hydroxide in TE buffer and 5 mL of heparin solution was added from a 2 mg/mL solution. This aqueous solution was added to an organic phase solution containing PLGA (lactide: glycolide 75:25) dissolved in Dichloromethane (DCM) at a 1:1 volume ratios (e.g. 2 mL of FGF2 solution and 2 mL of PLGA solution were added to a tube). This solution was then vortexed to create an emulsion. Next, 3 mL of 0.5% of polyvinyl alcohol (PVA) in water was added to produce a water/oil/water emulsion and this was repeated 2 additional times. Finally, the solution was added to a large volume of 0.5% PVA solution (200 mL) to remove aqueous phase and harden PLGA microbeads. The microbeads were then isolated by centrifugation (1500 rpm, 3 min) and washed 3 times with distilled water. Microbead preparation resulted in 120 mL total volume of bead solution at a bead concentration of about 2.5×10⁶ beads per mL.

The GF release level from microbeads can vary and therefore was determined empirically. The FGF2 release level from microbeads was determined by adding 8 μL of beads (about 20,000 beads) into 1 mL of medium in a 24 well plate. The plate was transferred to a cell culture incubator set at 37° C. 70 μL samples of the medium were taken at 24, 48 and 72 hours. The FGF2 level in the medium was measured by ELISA (cat #DFB50, R&D Biotechne Minneapolis, Minn.) or using a flow cytometry based FGF2 FlexSet (cat #BD 558327, BD Bioscience Franklin Lakes, N.J.). The average FGF2 release level over 24-72 hours was about 20 ng/mL when 8 μL of bead solution was added into 1 mL of medium.

StemBeads FGF2® purchased from StemCultures LLC when concentrated 2-fold brought about similar effects. StemCultures LLC released about 10 ng/mL when 8 μL of beads was added into 1 mL of medium. StemBeads FGF2® were concentrated by taking 10 mL of the bead suspension and centrifuging. Then, 5 mL (50% of the volume) of the liquid above the beads was removed (no beads were removed), thus concentrating the StemBeads FGF2® 2-fold.

Next, the PEGDA-hydrogel materials were prepared. 1.2 g of polyethylene glycol diacrylate (PEGDA, Laysan Bio Arab, AL, cat #ACRL-PEG-ACRL-20K-5g) was weighed out and the powder was transferred into a 15 mL conical tube. 5.4 mL of PBS (Gibco, 14190-144) was added to dissolve the PEG monomers. 600 μL of a 200 mM stock solution of the photo initiator LAP (Tocris Bio-Techne Minneapolis Minn., Cat. No 6146) dissolved in PBS was added to achieve a 2×-working concentration of LAP and PEGDA: 20 mM of LAP and 20% weight by volume PEGDA. This PEGDA-LAP solution was sterilized by passing it through a syringe filter with a 0.22 μm filter. After filtering, an equal volume of the StemBeads FGF2® solution was added (e.g. 6 mL of PEG solution was added to 6 mL of StemBeads FGF2®). The PEGDA-LAP-StemBeads® solution was mixed thoroughly and transferred to a reagent reservoir. 8 μL or 16 μL droplets were dispensed into non-treated cell culture plastic dishes.

The dishes containing 16 μL droplets were exposed to UV light (wavelength 390 nm, power 80 mW/cm²) for 30 seconds to polymerize the hydrogel and encapsulate the FGF2-StemBeads®. The dishes containing 8 μL droplets were exposed to UV light (wavelength 390 nm, power 80 mW/cm²) for 15 seconds to polymerize the hydrogel and encapsulate the FGF2-StemBeads®. The pipetting step was repeated until all the PEGDA-LAP-StemBeads® solution has been used to make droplets and all droplets have been exposed to UV. The diameter of each 16 μL hydrogel support was between about 2-3 mm and each was between about 0.2-0.5 mm thick after polymerization. These dimensions increased 2-3 times after the FGF2 feeding devices were added to culture medium and fully hydrated. 5 mg of FGF2 protein yielded about 15,000 feeding devices of 16 μL size hydrogel supports with an average release level of 10 ng/mL of FGF2 when added to 2 mL of medium.

Feeding devices were made at two different sizes and added into different volume of medium resulted in the same FGF2 release concentration. One 16 μL sized feeding device was added to 2 mL of medium and compared to one 8 μL sized feeding device was added to 1 mL of medium. FGF2 release levels were measured from medium samples collected over 7 days. Both sized feeding devices achieved an average release at the 10 ng/mL level (See FIG. 2C).

### Example 2: Feeding Device with an Alginate Hydrogel Support Preparation

This describes how to make FGF2 feeding devices using a polysaccharide alginate hydrogel support. Alginate can be chemically cross-linked via adding methacrylate groups to create a non-degradable hydrogel. Alginate is inert and does not support cell attachment. This method can be adjusted to make feeding devices of various types and amounts of GFs within hydrogel supports of various sizes and shapes.

Lyophilized recombinant FGF2 proteins is encapsulated into PLGA microbeads via double emulsion process as described in Example 1. The release level can vary and therefore is determined empirically, described in Example 1.

Next, the Alginate-hydrogel materials are prepared. 1.2 g of alginate methacrylate powder (Alginate-MA, Sigma-Aldrich St. Louis Mo.) is weighed out in a 15 mL conical tube. 5.4 mL of PBS is added to dissolve the alginate-MA monomers. 600 μL of a 200 mM stock solution of the photo initiator LAP (Tocris Bio-Techne Minneapolis Minn., Cat. No 6146) is dissolved in PBS and is added to achieve a 2×-working concentration of LAP and Alginate-MA: 20 mM of LAP and 20% weight by volume alginate-MA. This solution is sterilized by passing it through a syringe filter with a 0.22 μm filter. After filtering, an equal volume of the StemBeads FGF2 solution is added (e.g. 6 mL of PEG solution is added to 6 mL of StemBeads®). The Alginate-MA-LAP-StemBeads® solution is mixed thoroughly and transferred to a reagent reservoir. Using a multi-channel pipettor, 16 μL droplets are dispensed into non-treated cell culture plastic dishes. The dishes containing the droplets are exposed to UV light (wavelength 390 nm, power 80 mW/cm²) for 30 seconds to polymerize the hydrogel and encapsulate the StemBeads®. The pipetting steps are repeated until all the Alginate-MA-LAP-StemBeads® solution has been used to make droplets. The resulting feeding devices are disc shaped and about 2-3 mm in diameter. In all this makes about 750 devices.

### Example 3: Feeding Device with a Hyaluronic Acid (HA) Hydrogel Support Preparation

This describes preparation of FGF2 feeding devices using a hyaluronic acid (HA) hydrogel support. HA that is chemically cross-linked creates a non-degradable hydrogel and HA does not support cell attachment (i.e. HA is inert).

Lyophilized recombinant FGF2 proteins are encapsulated into PLGA microbeads via double emulsion process as described in Example 1. The release level can vary and therefore is determined empirically, as for example described in Example 1.

Next, the HA hydrogel materials are prepared. First, 100 mg of HAMA powder (PhotoHA-Stiff, #5275-1KIT, Advanced BioMatrix 5930 Sea Lion Pl, Carlsbad, Calif. 92010) will be weighed out in a 15 mL conical tube. 5 mL of PBS is added to dissolve the HAMA monomers, followed by 5 mL of FGF2-StemBead solution, Next 200 μL of photo initiator ruthenium solution is added and mixed to the HAMA solution from a stock solution of 37.4 mg/mL of ruthenium in PBS. 200 μL of sodium persulfate is added from a stock solution of 119 mg/mL of sodium persulfate in PBS. The HAMA-LAP-StemBeads® solution is mixed thoroughly and transferred to a reagent reservoir. Using a multi-channel pipettor, 16 μL droplets are dispensed into non-treated cell culture plastic dishes. The dishes containing the droplets are exposed to visible light (wavelength 400-450 nm) for 15 minutes for crosslinking. The feeding devices have a disc shape and are about 2-3 mm in diameter. In all this makes about 625 devices.

### Example 4: FGF2 Feeding Device with a Polyacrylamide (PA) Hydrogel Support Preparation

This describes preparation of FGF2-feeding devices from a polyacrylamide hydrogel support. This example demonstrates feeding devices can be made with a different type of polymerization and a different non-degradable hydrogel material. This example also demonstrates a feeding device that releases at very low concentrations (less than 1 ng/mL) which can mimic in vivo GF levels.

Lyophilized recombinant FGF2 proteins were encapsulated into PLGA microbeads via double emulsion process as described in Example 1. Next, the polyacrylamide solution was prepared. Acrylamide solution (40%, 29:1 acrylamide:bis-acrylamide) was mixed with water and StemBeads® at a 1:1:1 volume ratio (e.g. 1 mL of acrylamide solution was added to 1 mL of water and 1 mL of StemBeads FGF2®). Then, 1% of APS (10% w/v) and 0.1% of TEMED was added (e.g. in a 1 mL of acrylamide-bead solution, 10 μL of APS and 1 μL of TEMED was added). This solution was mixed thoroughly and pipetted in between 2 glass slides with 0.5 mm spacers. This cassette with the solution was incubated at room temperature for about 1 hour to allow for free radical polymerization to occur. This allowed the hydrogel support to polymerize and encapsulate the beads. After polymerization, the gel was removed from the cassette. Using a biopsy punch, disc shaped pieces were cut out to form feeding devices. In this example, the feeding devices were 5 mm in diameter and 0.5 mm in thickness.

One feeding device was added into 500 μL of medium in a well of a 24-well plate. The 24-well plate was then placed in the cell culture incubator. Medium samples were taken at day 2 and day 7. The FGF2 released was measured using FGF2 ELISA (cat #DFB50, R&D Biotechne Minneapolis, Minn. 55413). The average FGF2 level was 206.4+/−6 pg/mL (n=2 feeding devices, n=2 timepoints).

### Example 5: Lack of GF Burst Release when Beads are within a Hydrogel Support

This example illustrates the lack of a burst release by feeding with beads loaded within hydrogel supports. Burst release refers to the initial fast release of a significant fraction of a payload after delivery of the payload (here GF's) into the release medium. The burst effect is an undesired event in controlled release technologies but an often-unavoidable outcome. Herein, the burst effect is defined as a short burst of high concentrations of GF released after the initial delivery of the payload (GFs) into the culture medium. This example compares two methods for controlled release of FGF2 into culture medium. A first method uses StemBeads FGF2® (no support) dispensed into a basal culture medium containing DMEM (cat #10313-021, Gibco) and FBS (cat #A38400-01, Gibco) to release FGF2 over 24 hours. The second method employs an FGF2-hydrogel feeding device, comprised of StemBeads FGF2® encapsulated into a PEG hydrogel support as disclosed in Example 1 above.

For the first method, 16 μL of StemBeads FGF2® (about 20,000 beads) were pipetted into one well of a 24-well plate containing 2 mL of medium.

In the second method, one FGF2 feeding device (which contains about 20,000 StemBeads FGF2® within a PEG hydrogel support) was incubated for 30 minutes in the same culture medium as used in the first method above, at room temperature and then placed into a different well of a 24-well plate containing 2 mL of the same medium. The 24-well plate was then placed in the cell culture incubator. Medium samples were taken at 1 hour, 5 hours and 24 hours.

All samples were measured for FGF2-release levels using an FGF2 BD Flexset (cat #BD 558327, BD Bioscience Franklin Lakes, N.J.).

StemBeads FGF2® (first method) released a 1.86-fold greater amount of FGF2 at 1-hour time-point and a 1.35-fold greater amount of FGF2 at 5-hour time-point compared to the level of FGF2 release achieved at 24 hours. This higher amount of FGF2 within the first hours of feeding with StemBeads FGF2® was characteristic of a burst effect. Conversely, StemBeads FGF2® loaded into a hydrogel support (second method) released a 70% at 1-hour and then 90% at 5-hour timepoints of the FGF2 level achieved at the 24-hour timepoint. This gradual increase in FGF2 level within the first hours of feeding with FGF2 feeding device demonstrated a lack of FGF2 burst. (See FIG. 3A)

It was surprising and unexpected that StemBeads FGF2® when loaded in a hydrogel support were able to avoid burst release.

### Example 6: Increased Longevity of GF Sustained Release when Beads are within a Hydrogel Support

This example illustrates the improvement in longevity of sustained release achieved by loading microbeads in a hydrogel support. Longevity of sustained release refers to the ability to sustain the GF level of the payload (here GF's) over extended time periods. This example compares two methods for controlled release of FGF2 into culture medium. The first method uses StemBeads FGF2® (no support) dispensed into a basal culture medium containing DMEM (cat #10313-021, Gibco) and FBS (cat #A38400-01, Gibco) to release FGF2 over 14 days. The second method employs an FGF2-hydrogel feeding device, comprised of StemBeads FGF2® encapsulated into a PEG hydrogel support that was made as disclosed in Example 1 above.

For the first method, 16 μL of StemBeads FGF2® (about 20,000 beads) were pipetted into one well of a 24-well plate containing 2 mL of medium.

In the second method, one FGF2 feeding device was added directly into a well of a 24-well plate containing 2 mL of the same medium used in the first method. GF release by the first and second methods were compared for longer time points; thus medium samples were taken at day 1, day 4, day 7, day 10 and day 14.

All samples were measured for FGF2 release levels using an FGF2 ELISA (cat #DFB50, R&D Biotechne Minneapolis, Minn.).

Over the 2 weeks after the deposit into culture medium, the FGF2 feeding device (second method) sustained FGF2 levels better than StemBeads FGF2® (first method). By day 4 and onward, the FGF2 feeding device (second method) maintained FGF2 levels significantly better than StemBeads FGF2® (first method). By day 7, the FGF2 feeding device was releasing around 60% of the day 1 GF levels whereas StemBeads FGF2® were releasing about 30% of day 1 GF levels. By day 14, the FGF2 feeding device was releasing around 45% of day 1 GF levels whereas StemBeads FGF2® was releasing about 20% of day 1 GF levels (See FIG. 3B).

This example establishes that the FGF2 feeding device improves the controlled release of FGF2 between 1 and 2 weeks by 2-fold. Conclusion; The non-biodegradable hydrogel supports disclosed herein can prolong and stabilize the release of FGF2 from the encapsulated FGF2-degradable beads into culture medium.

### Example 7: Feeding Device Added to Different Volumes of Medium Results in Different GF Levels

This example shows how feeding devices can be added to different volumes of medium to achieve different levels of GF.

16 μL sized FGF2 feeding devices were prepared following methods described in Example 1 and set to release 10 ng/mL of FGF2 in 1 mL of culture medium.

For the first method, one FGF2 feeding device was dispensed into one well of a 24-well plate containing 1 mL of medium (a basal culture medium containing DMEM (cat #10313-021, Gibco) and FBS (cat #A38400-01, Gibco)). For the second method, one FGF2 feeding device was dispensed into one well of a 24-well plate containing 2 mL of the same medium. For the third method, one FGF2 feeding device was dispensed into one well of a 24-well plate containing 3 mL of the same medium.

Next, the well plate was placed in a cell culture incubator at 37° C. for one week. FGF2 levels were measured from medium samples collected over the 7 days using an FGF2 ELISA. This example demonstrated a linear relationship between FGF2 release level and the volume of culture medium into which a device is deposited into (See FIG. 4A).

### Example 8: Hydrogel Supports Loaded with Different Amounts of GF-Releasing Beads Results in Different GF Levels

This example demonstrates preparation of 16 μL sized (2-3 mm diameter disc shaped supports) feeding devices that release different levels of GF when added to the same volume of medium.

For the first method, 16 μL sized FGF2 feeding devices were manufactured following methods described in Example 1. This yielded hydrogel supports about 2-3 mm in diameter and contained about 20,000 StemBeads FGF2®. These feeding devices released an average of about 20 ng/mL FGF2 over 7 days when added to 1 mL of culture medium at 37° C. measured using flow cytometry with a FGF2 FlexSet (cat #BD 558327, BD Bioscience Franklin Lakes, N.J.).

In the second method, 16 μL sized FGF2 feeding devices (about 2-3 mm in diameter) were manufactured using the methods described in Example 1 but with a concentrated StemBeads® solution. To concentrate the StemBeads® solution, 100 mL of the StemBeads FGF2® solution (microbeads suspended in aqueous solution) was centrifuged and then 80 mL of the liquid above the beads was removed (no beads were removed), thus concentrating the bead solution 5-fold. This yielded hydrogel supports containing about 100,000 StemBeads FGF2®. These feeding devices released an average of about 100 ng/mL FGF2 over 7 days when added to 1 mL of culture medium at 37° C. measured using flow cytometry with a FGF2 FlexSet (cat #BD 558327, BD Bioscience Franklin Lakes, N.J.).

These two methods of making feeding devices resulted in the same size and shaped devices (2-3 mm diameter discs) but yielded different FGF2 release levels when added into the same volume of culture medium, (See FIG. 4B).

### Example 9: Feeding Devices Containing EGF and FGF2 Releasing Beads within a PEG Hydrogel Support

This example describes the creation of a hydrogel support loaded with multiple different GFs. One application of this type of feeding device is to replace fetal bovine serum (FBS). FBS contains a variety of GFs and stabilizing agents that have a potent influence on cell behavior. In this example, FGF2 and EGF were loaded into a single hydrogel support. This feeding device had a 16 μL volume size and was disc shaped (diameter of 2-3 mm) and had a controlled release of GFs: 10 ng/mL of EGF and 10 ng/mL of FGF2 when added to 1 mL of medium.

Lyophilized recombinant GF proteins were encapsulated into PLGA microbeads via double emulsion process as described in Example 1. The average release level over 24-72 hours for 8 μL of StemBeads FGF2® was 20 ng/mL and 10 μL of StemBeads EGF® was 20 ng/ml. The PEGDA-hydrogel materials were prepared as described in Example 1.

Next, one part PEG-LAP solution was mixed with 0.5 parts StemBeads FGF2® and 0.5 parts StemBeads EGF® (e.g. 2 mL of PEG solution was added to 1 mL of StemBeads FGF2® and 1 mL of StemBeads EGF®). 16 μL droplets of the combined solution were dispensed into non-treated cell culture plastic dishes as described in Example 1. In all about 225 16 μL sized (2-3 mm diameter discs) feeding devices were created.

One EGF and FGF2 combination feeding device was added into 1 mL of medium and placed at 37° C. for one week. Medium samples were collected on day 1, day 5 and day 7 and measured for EGF and FGF2 levels using an EGF ELISA (cat #DEG00, R&D Biotechne Minneapolis, Minn.) and an FGF2 ELISA (cat #DFB50, R&D Biotechne Minneapolis, Minn.). The average release of FGF2 over the 7 days was 8.1 ng/mL and the average release of EGF over the 7 days was 9.5 ng/mL (n=3 devices, n=3 time points) (See FIG. 5A).

### Example 10: Colored Feeding Devices with a PEG Hydrogel Support Preparation

This describes how to make colored feeding devices using PEG-hydrogel supports. This colored feeding device is 16 μL volume size and contains controlled release GFs. This method can be adjusted to make feeding devices of various types and containing various quantities s of GFs within hydrogel supports of different sizes, shapes and colors (red, green, yellow etc.).

Lyophilized recombinant GF proteins are encapsulated into PLGA microbeads via double emulsion process and PEGDA-hydrogel materials are prepared as described in Example 1.

Next, one part PEG-LAP solution is mixed with 0.5 parts StemBeads FGF2® and 0.5 parts colored beads (e.g. 2 mL of PEG solution is added to 1 mL of StemBeads FGF2® and 1 mL of yellow-colored beads). 16 μL droplets are dispensed into non-treated cell culture plastic dishes and polymerized as described in Example 1.

One feeding device containing StemBeads FGF2® and red-colored beads and one feeding device containing StemBeads EGF® and yellow-colored beads are added into a single well of a 6-well plate. These distinguishable feeding devices can be removed at different times from the culture, e.g. the FGF2 red feeding device is removed with a fine tip tweezer from the culture after 3 days of culture and the EGF yellow feeding device is removed after 1 week. This effectively removes FGF2 from the culture dish on day 3 without requiring a medium change and without removing EGF. This cannot be achieved using StemBeads® without a hydrogel support nor soluble GFs.

### Example 11: Magnetic FGF2 Feeding Device with a PEG Hydrogel Support

This describes how to make magnetic feeding devices to assist in the addition to, removal from and location of GFs within cell culture vessels. In the example, the feeding device is 16 μL volume size (disc shaped—diameter 2-3 mm).

Lyophilized recombinant GF proteins were encapsulated into PLGA microbeads via double emulsion process and PEGDA-hydrogel materials were prepared as described in Example 1. Then, one-part PEG-LAP solution was mixed with 0.5-part StemBeads FGF2® and 0.5-part magnetic beads (e.g. 2 mL of PEG solution was added to 1 mL of StemBeads FGF2® and 1 mL of magnetic agarose beads (20-60 μm in diameter, cat #130-093-657 Cube Biotech Wayne, Pa.). 16 μL droplets were dispensed into non-treated cell culture plastic dishes and droplets polymerized as described in Example 1. This procedure made about 225 16 μL sized disc shape (2-3 mm diameter) feeding devices.

One FGF2-feeding device containing magnetic beads was inserted into a culture dish with 2 mL of medium and placed in a cell culture incubator at 37° C. The FGF2 release from the magnetic feeding device was measured from medium samples taken at day 1, day 3 and day 6 using an FGF2 ELISA (cat #DFB50, R&D Biotechne Minneapolis, Minn.). An average release was 5.8 ng/mL (n=2 devices, n=3 time points) (See FIG. 5B). The magnetic feeding device was removed from the culture dish using a magnet attached to the end of a thin plastic rod (also known as a stir bar retriever). This effectively removed FGF2 from the culture dish.

### Example 12: Floating FGF2 Feeding Device with a PEG Hydrogel Support

This describes how to make feeding devices that float near the top of the cell culture medium to prevent feeding devices interfering with cells growing at the bottom of the plate. In the example, the feeding device is 16 μL volume size and disc shaped with a diameter of 2-3 mm (prior to swelling).

Lyophilized recombinant GF proteins were encapsulated into PLGA microbeads via double emulsion process and PEGDA-hydrogel materials were prepared as described in Example 1.

Then, one-part PEG-LAP solution was mixed with one-part StemBeads® (e.g. 2 mL of PEG solution was added to 2 mL of StemBeads FGF2®). 16 μL droplets were dispensed into non-treated cell culture plastic dishes. Then the solution was triturated (e.g. pipetting up and down in the bead-hydrogel solution several times) with a 200 μL pipette tip that contained air (the pipette tip did not contain solution) to introduce air bubbles. After the air bubbles were visible in the droplet, the droplets were exposed to UV light (wavelength 390 nm, power 80 mW/cm²) for 30 seconds to polymerize the hydrogel with bubbles and encapsulate beads.

One floating FGF2 feeding device was inserted into a culture dish with 2 mL of medium using sterile forceps and placed in the cell culture incubator at 37° C. The device remained near the surface of the medium and FGF2 release from the floating device was from medium samples collected over 6 days using an FGF2 ELISA kit (cat #DFB50, R&D Biotechne Minneapolis, Minn.). The average FGF2 release from the floating feeding device was 10.9 ng/mL (n=2 devices, n=3 time-points), (See FIG. 5C).

### Example 13: Tethered Feeding Device Made of PEG Hydrogel Support

This describes how to make hydrogel feeding devices that have a tether to assist in adding and removing from cell culture. A piece of sterile non-biodegradable suture made of nylon (Medtronic) about 3 inches long (length can vary based on application) was placed onto a non-tissue treated dish. The suture (intended to act as a tether) was laid in a straight line. Next, a 25 μL droplet (prepared as in Example 1) was dispensed at one end of the suture, with the suture end located in the middle of the droplet. The droplet was exposed to UV light (wavelength 390 nm, power 80 mW/cm²) for 30 seconds to polymerize the hydrogel around the end of the suture and encapsulate the GF releasing beads. One tethered feeding device made as set forth above was inserted into and subsequently removed from a culture dish by handling the tether.

### Example 14: Improved Pluripotency of iPSCs Using an FGF2 Feeding Device Compared to Conventional Culture Method (Daily Feeds of Soluble FGF2)

iPSC lines from different donors can vary greatly, including how easy or difficult they are to maintain in the pluripotent (undifferentiated) state. In this example, two methods of maintaining iPSCs in culture across iPSC lines derived from different donors that exhibit different ease of culture using conventional methods were compared. This example demonstrates using FGF2 feeding devices was superior in maintaining iPSC lines in an undifferentiated state compared to conventional culture methods using daily feeding of soluble FGF2, specifically for iPSC lines that were difficult to maintain in a pluripotent state. This example shows that using a feeding device uses less medium during iPSC culture and yet still improves pluripotency of iPSCs across lines.

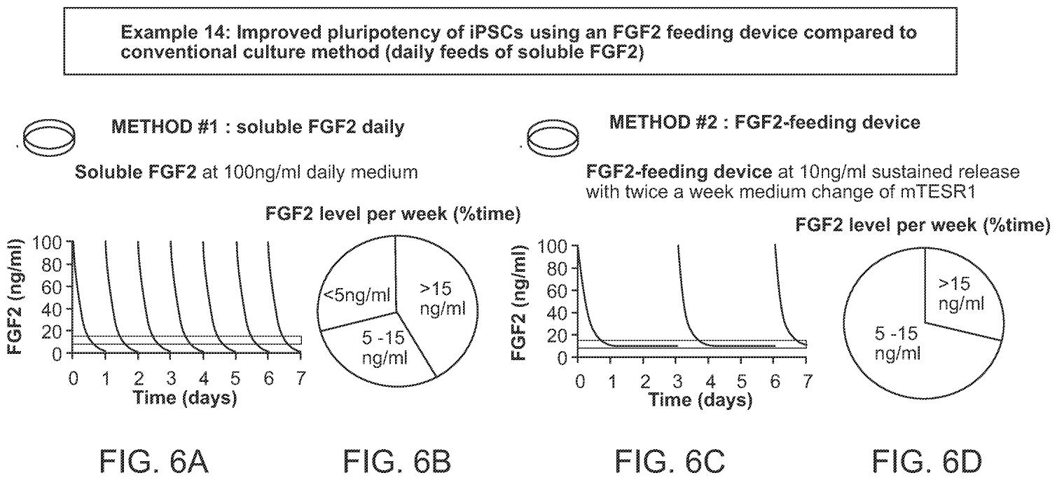

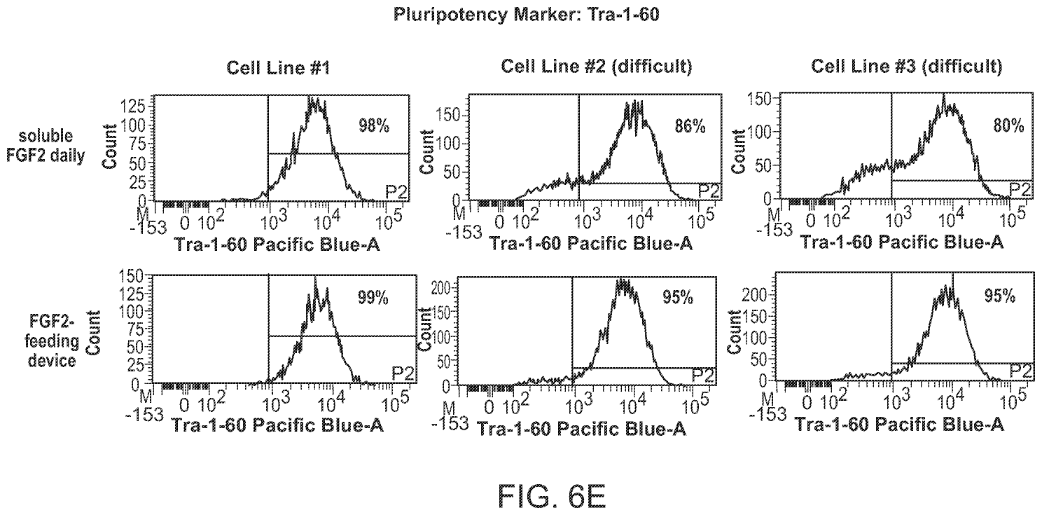

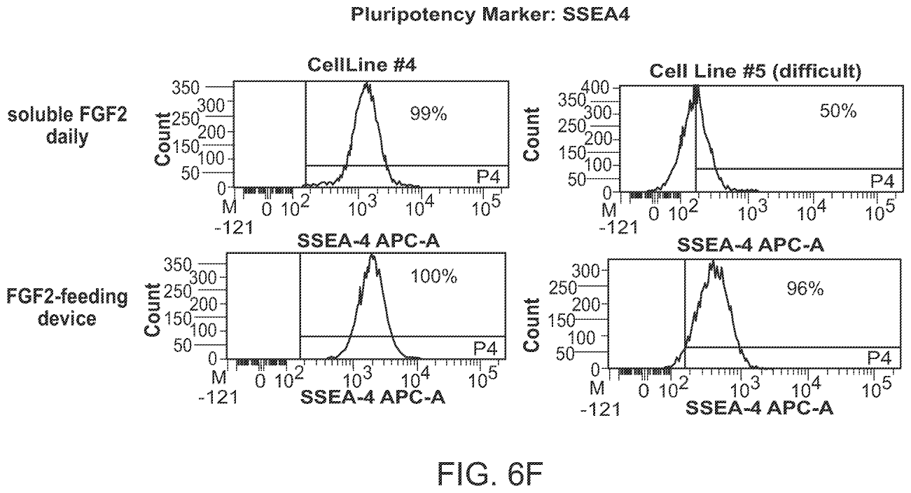

In the first method, iPSCs were cultured in the conventional method of daily medium exchanges of mTESR1 (containing 100 ng/mL of soluble FGF2). Each well contained 2 mL of mTESR1 medium and was replaced with fresh medium daily which totals 14 mL of medium used per week per well. FGF2 level fluctuated daily (See FIG. 6A) and cells spent about a third of the time with FGF2 levels less than 5 ng/mL per week, since soluble FGF2 has a half-life of about 4 hours (See FIG. 6B).

In the second method, iPSCs were fed using FGF2 feeding devices added to mTESR1 medium. Each well contained 2 mL of mTESR1 medium and one FGF2 feeding device (method described in Example 1). The medium was replaced with 2-3 times per week and the feeding device was replaced once a week. This totals 4-6 mL of medium was used per week per well. FGF2 level fluctuates less often due to the reduced medium exchanges compared to the first method (See FIG. 6A compared to FIG. 6C) and cells spend no time with FGF2 levels less than 5 ng/mL per week (See FIG. 6D).

3 iPSC lines (cell line #1, F11350.1, cell line #2 FA14530.1-d1E02, cell line #3 FA14530.1-d1G12) were thawed onto Matrigel coated 6-well dishes and cultured in both methods, described above. iPSCs were passaged about once a week using ReLeSR (Stem Cell Technologies). After 4 passages (about 4 weeks of culture) using these two different culturing methods, cells were grown to about 60-80% confluence and collected for flow cytometry to quantitatively measure expression levels of pluripotent markers such as Tra-1-60.

In a repeat experimental set up, 2 additional iPSC lines (cell line #4, GIH-7-d2d2B12 and cell line #5 GIH-7-C2) were used and cultured following the same methods described above. Similarly, after 4 passages cells were collected for flow cytometry to quantitatively measure expression levels of pluripotent markers such as SSEA4.

### Results

Cell line #1 maintained high levels of pluripotency marker Tra-1-60 in both methods. Cell line #2 and #3 (both difficult to maintain in the pluripotent state), improved pluripotency when cultured with less frequent medium changes and FGF2 feeding devices (second method). Cell lines #2 and #3 cultured by the conventional method of daily mTESR1 medium changes (first method) measured 80-86% cells positive for pluripotency marker Tra-1-60. This improved to 95% Tra-1-60 positive cells when these lines were cultured with less frequent medium changes and FGF2 feeding devices (second method), (See FIG. 6E).

In the repeat experiment, cell line #4 maintained high levels of pluripotency marker SSEA4 in either method. Cell line #5 (difficult to maintain in the pluripotent state) improved pluripotency when cultured with FGF2 feeding devices (second method). When cell line #5 was cultured with daily mTESR1 (first method), 50% of cells were positive for SSEA4. This improved to 96% of cells positive for SSEA4 when cell line #5 was cultured with less frequent medium changes and FGF2 feeding devices (second method) (See FIG. 6F).

This example establishes that the FGF2 feeding device improves maintenance in the pluripotent state of iPSC lines that are difficult to keep in an undifferentiated state despite a 3-fold lower use of medium per week when iPSCs were cultured with FGF2 feeding devices (second method) compared to the conventional mTESR1 daily feds (first method).

### Example 15: Using an FGF2 Feeding Device to Grow iPSCs Results Improved Mesoderm Differentiation Compared to Daily Soluble FGF2, Stabilized FGF2 or FGF2-Releasing Microbeads

This example compares 5 methods of growing iPSCs to subsequently make mesoderm cells. This example compares growing iPSCs with (1) first method—soluble FGF2 using mTESR1 medium, (2) second method—FGF2-feeding device with mTESR1 medium (3) third method stabilized soluble FGF2 using mTESR1-Plus medium (Stem Cell Technologies), (4) fourth method—FGF2 feeding device with mTESR1-Plus medium and (5) fifth method-StemBeads FGF2® with mTESR1 medium, and measures how well iPSCs cultured in these different methods can make quality mesoderm brachyury progenitor cells (2D, mesoderm lineage). The example demonstrates that using a feeding device requires less medium and less FGF2 during iPSC culture yet still improves iPSCs ability to be differentiated into mesoderm cells compared to iPSCs cultured with soluble FGF2, stabilized soluble FGF2 or StemBeads® alone (no hydrogel support).

In the first method, soluble GFs were used to grow iPSCs by changing the medium daily (no beads, no support). The medium used was mTESR1 which contains 100 ng/mL of soluble FGF2. 2 mL of medium was used daily, a total of 14 mL of medium was used per week per well.

In the second method, cells were grown using one FGF2 feeding device containing about 20,000 StemBeads FGF2® (refer to Example 1 for methods) added once per week and 2 mL of mTeSR1 medium was added fresh twice a week per well (feeding device was not removed during medium exchanges). A total of 4 mL of medium was used per week per well.

In the third method, mTESR1-Plus medium was used to grow the iPSCs. In this condition, iPSCs were grown in mTESR1-Plus following the manufacturing protocol: 2 mL medium change on Mondays and Wednesdays and a 4 mL medium change on Fridays. A total of 8 mL of medium was used per week per well.

In the fourth method, iPSCs were grown using one FGF2-feeding device containing about 20,000 StemBeads FGF2® (refer to Example 1 for methods) added once per week and 2 mL of mTESR1-Plus was added fresh after iPSC passage and once mid-week (feeding device was not removed during the medium exchange). A total of 4 mL of medium was used per week per well.

In fifth method, iPSCs were grown by adding about 20,000 StemBeads FGF2® (no hydrogel support) into the medium. Both mTESR1 medium and StemBeads FGF2® were exchanged on Mondays, Wednesdays and Fridays. To replace StemBeads FGF2®, the culture well was first washed 2 times with DMEM-F12 to remove old beads before fresh 2 mL of mTESR1 and fresh StemBeads FGF2® were added. StemBeads FGF2® were added at a concentration that maintains a relatively uniform level of 10 ng/mL of FGF2 (the same controlled release level of FGF2 achieved from the FGF2 feeding device used in this example). A total of 6 mL of medium was used per week per well.

iPSC lines derived from different donors were thawed into Matrigel coated 6-well plate and grown following the five different iPSC culture methods described above. All culture methods were passaged about once a week using ReLeSR (Stem Cell Technologies). In methods that used a feeding device, the feeding device was replaced once a week after passaging the iPSCs. After 4 passages (approximately 4 weeks of culture), iPSCs grown with each culture method were single cell harvested and plated at 95-100% confluency into a Matrigel-coated 96 well plate. The next day, mesoderm differentiation medium (Stem Cell Tech, cat #05233) was added to all wells. At the 30-hour time-point, mesoderm progenitor cells were harvested, RNA was isolated and cDNA was generated for qPCR. Brachyury (which has the gene symbol ‘T’), a positive marker for progenitor mesoderm cells was measured via qPCR.

### Results

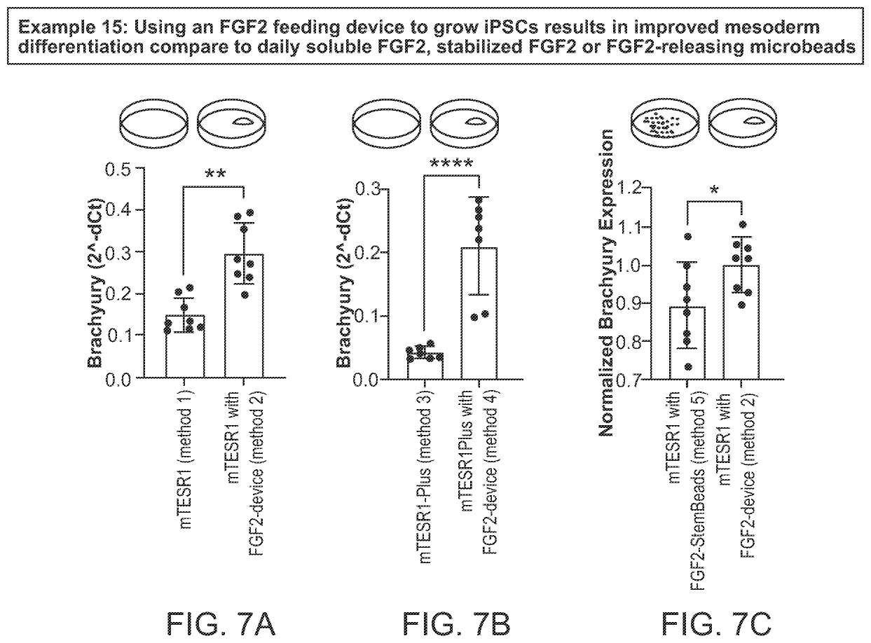

Mesoderm cells generated from iPSCs grown with an FGF2 feeding device with mTESR1 (method #2) had a 2.1-fold increase in brachyury expression compared to mesoderm cells generated from iPSCs cultured with daily mTESR1 medium changes (method #1) (See FIG. 7A). Note using mTESR1 daily in method #1 required 3.5-fold more medium compared to using a feeding device delivered with mTESR1 medium (method #2).

Mesoderm cells generated from iPSCs grown with an FGF2 feeding device with mTESR1-Plus (method #4) had a 4.7-fold increase in brachyury expression compared to mesoderm cells generated from iPSCs cultured with mTESR-Plus alone (method #3) (See FIG. 7B). Note using mTESR1-Plus in method #3 required 2-fold more medium compared to using a feeding device delivered with mTESR1-Plus medium (method #4).

Mesoderm cells generated from iPSCs grown with an FGF2 feeding device with mTESR1 (method #2) had a 1.1-fold increase in brachyury expression compared to mesoderm cells generated from iPSCs cultured with StemBeads FGF2® (no hydrogel support) with mTESR1 (method #5) (See FIG. 7C). Note using StemBeads® alone (no hydrogel support) delivered with mTESR1 medium in method #5 required 1.5-fold more medium compared to using a feeding device delivered with mTESR1 medium (method #2). Also note using StemBeads FGF2® alone (no hydrogel support) in method #5 required 3-fold more StemBeads FGF2® compared to methods using a feeding device (method #2 and #4).

Overall, higher-quality mesoderm cell fate was achieved from cultures originating from iPSCs grown with an FGF2-device in either mTESR1 or in mTESR1-Plus medium, as demonstrated by a higher gene expression level of the marker brachyury (T). It is surprising that across multiple iPSC media and even when a more stabilized FGF2 is included (mTESR1-Plus), the FGF2-feeding device is beneficial for quality cell differentiation across iPSC lines. The beneficial effect of FGF2 with a feeding device (method #2) over StemBeads FGF2® (method #5) is observed even though the feeding device uses ⅓ the number of StemBeads FGF2® per week.

### Example 16: FGF2 Feeding Device Delivered with Medium without Soluble FGF2 for iPSC Culture Subsequently Improves Directed Differentiation of iPSCs into Endoderm, Mesoderm and Ectoderm Lineages

This example compares two methods of growing iPSCs to subsequently make endoderm, mesoderm and ectoderm cells. The example compares growing iPSCs by a conventional method of daily medium feeds of soluble FGF2 to using an FGF2 feeding device with less frequent medium changes of a medium without soluble FGF2. The initial quality of iPSC cultures determines the efficiency of subsequent differentiation into specific cell types. This example demonstrates iPSCs grown under conventional culture (with daily soluble FGF2-no support) method fail to efficiently differentiate into endoderm, mesoderm and ectoderm progenitor cells. However, when they were cultured with FGF2 feeding devices (no soluble FGF2) successfully increase directed differentiation efficiencies across all 3 germ layers even with less medium used and no soluble FGF2 added into the medium.

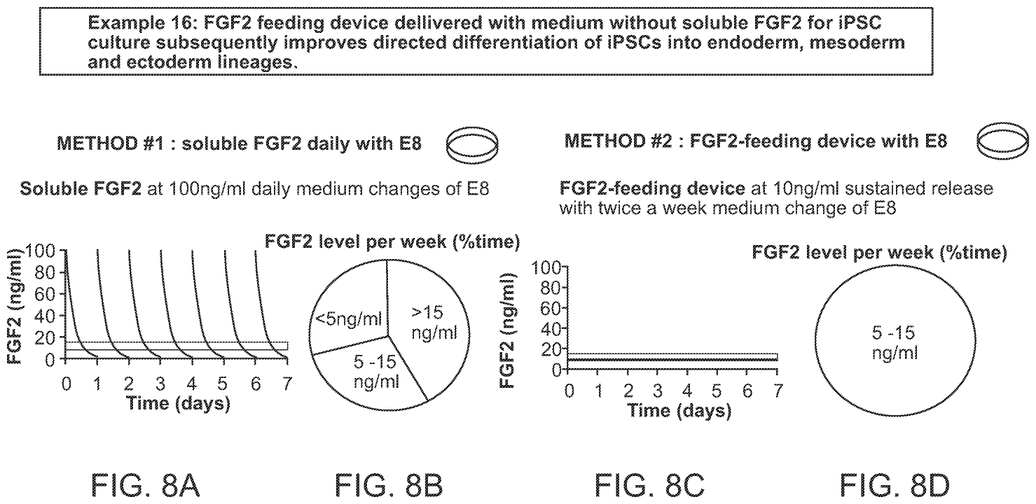

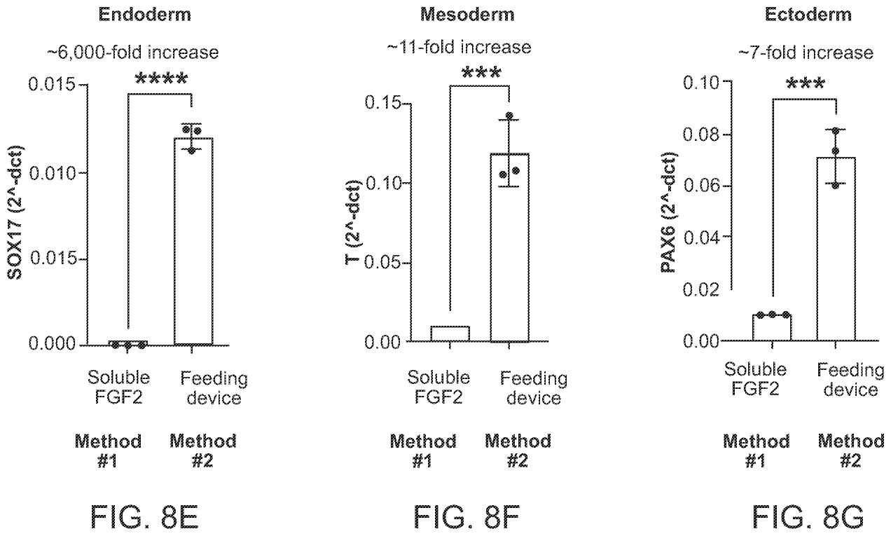

In the first method, soluble GFs were used to grow iPSCs by daily medium changes of Essential8 medium (E8, Gibco). E8 is made up of Essential6 medium (E6, Gibco) with soluble TGFb1 (2 ng/mL) and soluble FGF2 (100 ng/mL). When 2 mL of E8 medium was added daily, a total of 14 mL of medium was used per week per well. FGF2 level fluctuate daily (See FIG. 8A) and cells spent about a third of the time with FGF2 levels less than 5 ng/mL per week, since soluble FGF2 has a half-life of about 4 hours (See FIG. 8B).

In the second method, cells were grown using FGF2 feeding devices (refer to Example 1 for methods) delivered to E8 medium made up without soluble FGF2. E8 medium without soluble FGF2 was made up by adding soluble TGFb1 (2 ng/mL) to E6 medium. A total of 6 mL of medium used per week per well. FGF2 level was provided solely from the feeding device and thus the level was relatively uniform (See FIG. 8C). 100% of the time FGF2 levels were between 5-15 ng/mL, supplied by the FGF2 feeding device (See FIG. 6D).

iPSCs were thawed into Matrigel coated 6-well plate and grown following the two culture methods described above. iPSCs were passaged about once a week using ReLeSR (Stem Cell Technologies). After 4 passages (approximately 4 weeks of culture), iPSCs grown with each culture method were single cell harvested and plated at 95-100% confluence into a Matrigel-coated 96 well plate (9 wells were plated for each iPSC culture method, 18 wells total).

The next day, three wells that contained iPSCs cultured with soluble FGF2 (first method) and three wells that contained cells cultured with FGF2 feeding devices (second method) were fed with 150 μL of endoderm medium (Stem Cell Tech, Cat #05233). These wells were re-fed every 24 hours and after 72 hours of differentiation, the wells were harvested for RNA isolation and qPCR was carried out for gene analysis of the endoderm marker SOX17.

Three wells that contained iPSCs cultured with soluble FGF2 (first method) and three wells that contained cells cultured with FGF2 feeding devices (second method) were fed with 150 μL of mesoderm medium (Stem Cell Tech, Cat #05233) and re-fed every 24 hours. After 30 hours of differentiation, the culture wells were harvested RNA isolated and qPCR conducted for gene analysis of the mesoderm marker Brachyury (the gene symbol for Brachyury is ‘T’).

Three wells that contained iPSCs cultured with soluble FGF2 (first method) and three wells that contained cells cultured with FGF2 feeding devices (second method) were fed with 150 μL of ectoderm differentiation medium (Stem Cell Tech, Cat #05233) every 24 hours. After 6 days of differentiation, the culture wells were harvested for RNA isolation and qPCR for gene analysis of the ectoderm marker PAX6.

### Results

iPSC cultures grown with FGF2 feeding devices (no soluble FGF2) generated endoderm cultures with about 6,000-fold increase in SOX17 expression compared to endoderm cultures generated from iPSC cultures grown with daily feds of E8 medium (with soluble FGF2). (n=3 wells; unpaired t-test **** p<0.00005). (See FIG. 8E)

Mesoderm cells generated from iPSCs grown with an FGF2 feeding device (no soluble FGF2) had an 11-fold increase in mesoderm marker brachyury expression compared to mesoderm cells generated from iPSCs cultured with daily feds of E8 medium (with soluble FGF2). (n=1 cell line; n=3 wells; unpaired t-test *** p<0.0005). (See FIG. 8F)

Ectoderm cells generated from iPSCs grown with an FGF2 feeding device (no soluble FGF2) had a 7-fold increase in ectoderm marker PAX6 expression compared to ectoderm cells generated from iPSCs cultured with daily soluble feds of E8 medium (with soluble FGF2). (n=1 cell line; n=3 wells; unpaired t-test *** p<0.0005). (See FIG. 8G)

It was unexpected and surprising that iPSC cultures grown with an FGF2 feeding device generated endoderm, mesoderm and ectoderm cultures with improved efficiency compared to the conventional culture method which feeds without a support, despite the decrease in medium (2.3-fold less) to grow iPSCs.

### Example 17: Using an FGF2 Feeding Device to Grow iPSCs Results in Improved (Neuroectoderm) Cerebral Organoid Differentiation

This example compares two methods of growing iPSCs to then generate cerebral organoids. The example compares growing iPSCs with no feeding device and addition of daily soluble FGF2 (mTESR1 medium) to a method using less frequent mTESR1 feeds delivered with an FGF2 feeding device. The procedure measures how well iPSCs cultured using these two different methods can make quality cerebral organoids (3D, neuroectoderm lineage). The initial quality of iPSC cultures determines the efficiency of subsequent differentiation into specific cell types. iPSC lines grown using traditional protocols sometimes differentiate poorly into cerebral cortex organoids. This example demonstrates iPSCs lines that had been grown using the conventional method (no feeding device) failed to produce organoids, those iPSC lines now cultured with FGF2-feeding devices differentiated into cerebral organoids efficiently.

In the first method, iPSCs were cultured in the conventional method of daily medium exchanges of mTESR1 (containing 100 ng/mL of soluble FGF2)—no feeding device. Each well contained 2 mL of mTESR1 medium and was replaced with fresh medium daily, which totals 14 mL of medium was used per week per well. FGF2 level fluctuate daily (See FIG. 6A) and cells spent about a third of the time with FGF2 levels less than 5 ng/mL per week, since soluble FGF2 has a half-life of about 4 hours (See FIG. 6B).

In the second method, iPSCs were fed using FGF2 feeding devices added to mTESR1 medium. Each well contained 2 mL of mTESR1 medium and one FGF2 feeding device (method described in Example 1). The medium was replaced with 2-3 times per week and the feeding device was replaced once a week. This totals 4-6 mL of medium was used per well per week. FGF2 level fluctuates less often due to the reduced medium exchanges compared to the first method (See FIG. 6A compared to FIG. 6C) and cells spend no time with FGF2 levels less than 5 ng/mL per week (See FIG. 6D).

iPSCs (frozen at passage 18) were thawed and grown on Matrigel coated six well plates for 4-5 weeks and passaged approximately once per week (4-5 passages total). After 4-5 weeks and 4-5 passages, iPSCs grown with each method were then harvested for cerebral organoid production following published methods (See Bowles et al, Temple S. Cell. 2021 Aug. 19; 184 (17): (4547-4563). After 2 months of cerebral organoid differentiation and culture, organoids were evaluated for cerebral cortex neuron subtype markers including CTIP2 and TBR1.

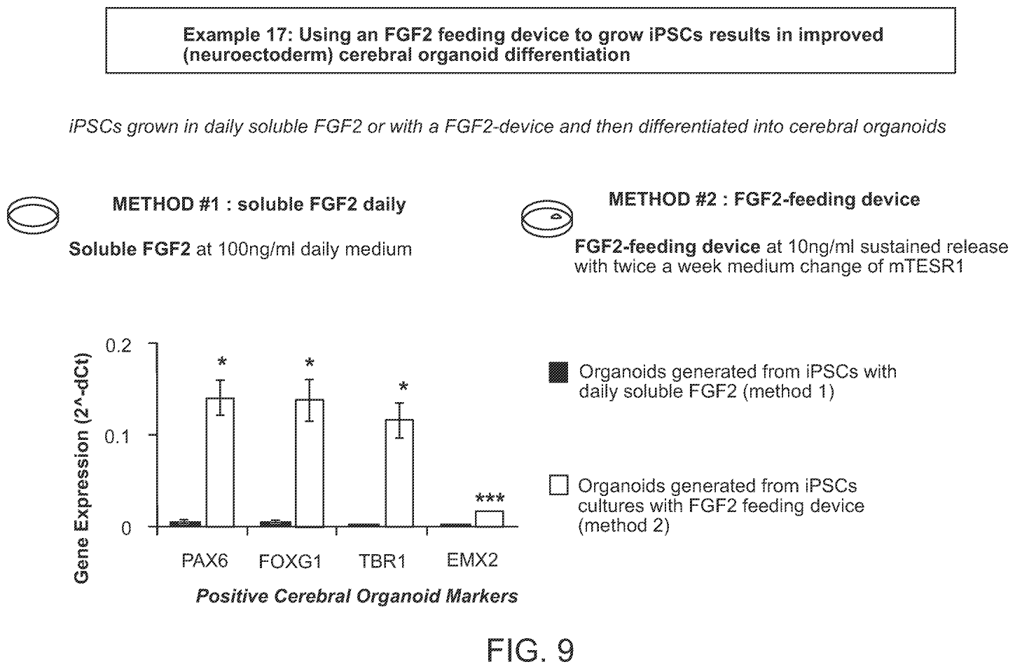

This example demonstrates that iPSC lines that previously failed to produce cerebral organoids after traditional iPSC growth (no feeding device) generated well-patterned cerebral cortex organoids after iPSC growth using an FGF2 feeding device. Organoids generated from iPSC cultured with an FGF2 feeding device (second method) demonstrated a 24-fold increase in gene expression of PAX6, a 27-fold increase in gene expression of FOXG1, a 145-fold increase in gene expression of TBR1 and a 23-fold increase in gene expression of EMX2 compared to organoids generated from iPSCs grown with daily medium changes (first method) (See FIG. 9). Hence, iPSC grown with the FGF2 feeding devices produced cerebral organoids surprisingly more efficiently than iPSCs grown with soluble GF despite the 3-fold less medium used to grow the iPSCs.

A number of embodiments have been described. Nevertheless, it will be understood that various modifications may be made without departing from the spirit and scope of the disclosure. Accordingly, other embodiments are within the scope of the following claims.

What is claimed:

1. A cell culture feeding device comprising a hydrogel polymer support, a plurality of microbeads within the support, the microbeads carrying cellular growth factors (GFs).

2. The support of claim 1 wherein the hydrogel polymer support is non-degradable.

3. The support of claim 2 wherein the polymer comprises PEG.

4. The support of claim 2 wherein the polymer comprises polyethylene glycol, polyacrylamide, pHEMA, alginate or hyaluronic acid.

5. The support of claim 2 wherein the support is biologically acceptable material.

6. The support of claim 5 wherein the microbeads comprise PLGA beads.

7. The support of claim 1 wherein the microbeads carry at least one GF member selected from the group consisting of FGF2, FGF4, FGF8, FGF18, WNT1, WNT3A, EGF, BDNF, GDNF, NT3, TGFb1, TGFb3, BMP2, BMP4, PDGFBB, PDGFAA, IGF1, VEGFA, VEGFC, SHH, cAMP, LIF, NRG1, SCF, ACTIVIN A, IL1b, IL2, IL6, IL7, IL12, IL15, IL21, IL34, IFNα, IFNγ, TAU, ABETA, A-SYNUCLEIN or modified versions.

8. The support of claim 1 wherein the support has been dried.

9. The support of claim 1 wherein one end of a tether is attached to the support.

10. The support of claim 1 further comprising magnetic particles.

11. The support of claim 1 further comprising a color.

12. The support of claim 1 containing gas bubbles to enable the support to float at or near the surface of cell culture media.

13. The support of claim 11 wherein the color is incorporated in a microbead.

14. A method of feeding a cell culture which comprises depositing an inert hydrogel polymer support into a cell culture media, the support carrying microbeads bearing one or more cellular GFs, the GFs being continuously released into the cell culture medium at a controlled rate over a period of time.

15. The method of claim 14 further comprising adjusting the quantity of microbeads in the support to deliver a predetermined quantity of GFs to the cell culture.

16. The method of claim 14 wherein the microbeads comprise PLGA beads.

17. A method of feeding a cell culture in cell culture media which comprises providing a hydrogel polymer support having a predetermined configuration and containing a plurality of microspheres carrying at least one GF and depositing the support into the cell culture media.

18. The method of claim 17 wherein the microspheres continuously release the GF into the cell culture media for at least 7 days.

19. The method of claim 17 wherein the support is labelled with a color.

20. The method of claim 17 wherein the culture media contains a plurality of supports, each support is colored and bears a different GF and a different color than the other supports.

21. The method of claim 17 wherein the support is attached to a tether.
